(19) United States
(12) Patent Application Publication
(10) Pub. No.: US 2002/0147175 A1
(43) Pub. Date: Oct. 10, 2002

(54) Synergistic ECTA compositions

(76) Inventors: H. Michael Shepard, Encinitas, CA (US); Christopher Boyer, San Diego, CA (US)

Correspondence Address: Antoinette F. Konski, McCutchen Doyle Brown & Enersen LLP, Three Embarcadero Center, Suite 1800, San Francisco, CA, 94111, US

(21) Appl. No.: 09/990,799

(22) Filed: Nov. 16, 2001

Related U.S. Application Data
(60) Provisional application No. 60/249,722, filed on Nov. 16, 2000.

(51) Int. Cl. A61K 31/7064; A61K 31/505; A61K 31/519; A61K 31/675

(52) U.S. Cl. 514/49; 514/256; 514/260.1; 514/86

(57) ABSTRACT

This invention provides compositions containing an effective amount of a novel substrate compound that selectively inhibit the proliferation of hyperproliferative cells, for example, pathological cells that endogenously overexpress a target enzyme that confers resistance to biologic and chemotherapeutic agents and an effective amount of a nucleoside transport antagonistic agents. Further provided by this invention is a method for treating a subject by delivering to the subject the composition as described herein. The compositions of this invention may be used alone or in combination with other chemotherapeutics or alternative anti-cancer therapies such as radiation.

### CROSS-REFERENCE TO RELATED APPLICATIONS

This application claims priority under 35 U.S.C. § 119(e) of U.S. Provisional Application No. 60/249,722, filed Nov. 16, 2000, the contents of which are hereby incorporated by reference into the present disclosure.

### TECHNICAL FIELD

The present invention relates to the field of drug discovery and therapy. Specifically, the present invention relates to the combination of antagonists of nucleoside transport agents and prodrugs that are substrates for overexpressed, endogenous intracellular enzymes.

### BACKGROUND OF THE INVENTION

Throughout and within this disclosure, various publications are referenced by first author and date, patent number or publication number. The full bibliographic citation for each reference can be found within the specification or at the end of this application, immediately preceding the claims. The disclosures of these publications are hereby incorporated by reference into this disclosure to more fully describe the state of the art to which this invention pertains.

Cancer is one of the most fatal human diseases worldwide. Treatment with anticancer drugs is an option of steadily increasing importance, especially for systemic malignancies or for metastatic cancers that have passed the state of surgical curability. Unfortunately, the subset of human cancer types that are amenable to curative treatment today is still rather small (Haskell, C. M. (1995)) resulting in about 600,000 deaths per year. See Cancer Facts & Figures, 1999 American Cancer Society. Progress in the development of drugs that can cure human cancer is slow, with success limited to a few hematological malignancies and fewer solid tumor types (Dorr, R. T. and Van Hoff, D. D. (1994)). Progress in discovering therapies that are based upon disease mechanism offers opportunities for future success. (Cobleigh, M. A. et al. (1999) and Roth, J. A. et al. (1999)).

The heterogeneity of malignant tumors with respect to their genetics, biology and biochemistry as well as primary or treatment-induced resistance to therapy mitigate against curative treatment. Moreover, many anticancer drugs display only a low degree of selectivity, causing often severe or even life threatening toxic side effects, thus preventing the application of doses high enough to kill all cancer cells. Searching for anti-neoplastic agents with improved selectivity to treatment-resistant pathological, malignant cells remains, therefore, a central task for drug development.

Cancer cells are characterized by uncontrolled growth, de-differentiation and genetic instability. The instability expresses itself as aberrant chromosome number, chromosome deletions, rearrangements, loss or duplication beyond the normal diploid number. (Wilson, J. D. et al. (1991)). This genomic instability may be caused by several factors. One of the best characterized is the enhanced genomic plasticity which occurs upon loss of tumor suppressor gene function (e.g., Almasan, A. et al. (1995a) and Almasan, A. et al. (1995b)). The genomic plasticity lends itself to adaptability of tumor cells to their changing environment, and may allow for the more frequent mutation, amplification of genes, and the formation of extrachromosomal elements (Smith, K. A. et al. (1995) and Wilson, J. D. et al. (1991)). These characteristics provide for mechanisms resulting in more aggressive malignancy because they allow tumors to rapidly develop resistance to natural host defense mechanisms, biologic therapies (See Wilson, J. D. et al. (1991) and Shepard, H. M. et al. (1988)), as well as to chemotherapeutics (See Almasan, A. et al. (1995a); and Almasan, A. et al. (1995b)).

In addition, the clinical usefulness of a chemotherapeutic agent may be severely limited by the emergence of malignant cells resistant to that drug. A number of cellular mechanisms are probably involved in drug resistance, e.g., altered metabolism of the drugs, impermeability of the cell to the active compound, accelerated drug elimination from the cell, altered specificity of an inhibited enzyme, increased production of a target molecule, increased repair of cytotoxic lesions, or the bypassing of an inhibited reaction by alternative biochemical pathways. In some cases, resistance to one drug may confer resistance to other, biochemically distinct drugs. An alternative mechanism of resistance to cancer chemotherapeutics occurs via the functional loss of tumor suppressor genes. The best characterized of these are p53, RB and p16. (Funk, J. O. 1999 and Teh, B. T. (1999)). Loss of function of these gene products leads to depressed expression of enzymes commonly targeted by anti-cancer drugs (e.g., 5-fluorouridyl (5FU)/thymidylate synthase and methotrexate/dihydrofolate reductase). (Lee, V. et al. (1997), Lenz, H. J. et al. (1998), and Fan, J. and Bertino, J. (1987)). Amplification of certain genes is involved in resistance to biologic and chemotherapy. Amplification of the gene encoding dihydrofolate reductase is related to resistance to methotrexate, while overexpression/amplification of the gene encoding thymidylate synthase is related to resistance to treatment with 5-fluoropyrimidines. (Smith, K. A. et al. (1995)).

Enzyme Catalyzed Therapeutic Activation (ECTA) was developed to circumvent drug resistance. One application of ECTA, TS ECTA, takes advantage of the overexpression of thymidylate synthase (TS) in many tumor cells. One TS ECTA compound, (E)-5-(2-bromovinyl)-2′-deoxy-5′-uridyl phenyl L-alaninylphosphoramidate (“NB 1011”) is a nucleotide analog phosphoramidate, which upon entry into cells is converted to bromovinyldeoxyuridine monophosphate (BVdUMP) (Lackey, D. B. et al. (2000)). Subsequently during an enzymatic reaction catalyzed by TS, BVdUMP is converted into proposed cytotoxic product(s) (Lackey, D. B. et al. (2000)). NB1011 is preferentially cytotoxic to tumor cells displaying elevated TS levels as compared to normal cells which have lower levels of TS. Furthermore, NB1011 was shown to have antitumor activity in colon and breast carcinoma xenografts in athymic mice (Lackey, D. B. et al. (2000)).

### DISCLOSURE OF THE INVENTION

The cytotoxicity of ECTA compounds in combination with selected chemotherapeutic agents with characterized mechanisms of action was investigated. Antagonists of nucleoside transporters were identified as a class of agents that preferentially enhance cytotoxicity of ECTA compounds on tumor cells. While not wishing to be bound to any theory, Applicants' results show that altering intracellular nucleoside pools via inhibition of transporter function dramatically increases the sensitivity of high TS expressing tumor cells to the cytotoxic effects of TS ECTA. Thus, while Applicants have specifically identified several compounds that are known to inhibit transporter function, any compound or therapy which produces the same result is believed to enhance the cytotoxicity of ECTA prodrugs. While others have noted enhanced activity of modified nucleosides in the presence of dipyridamole (Grem, J. L. (1992) and Wright, A. M. et al. (2000)), the synergistic activity reported herein with ECTA prodrugs is novel. This especially applies to the lack of synergistic toxicity on normal cells. The results reported herein also supports the theory that NB1011 is a nucleotide substrate of thymidylate synthase, as opposed to the classical inhibitors of TS function now in clinical use.

Thus, this invention provides a composition comprising an ECTA compound or prodrug wherein the ECTA prodrug is selectively converted to a toxin in the cell by an endogenous, intracellular target enzyme and a nucleoside transport inhibitor. Specific ECTA compounds for use in the composition are one or more selected from the group consisting of a 1,5-substituted pyrimidine; a substituted furanopyrimidone; 1,5-substituted pyrimidine; a pyrimidine substituted at the 5 position with a group that is extractable from pyrimidine by the endogenous, intracellular enzyme wherein the 5-substituent is selected from the group consisting of alkyl, alkenyl, alkynyl, vinyl, propargyl and substituted derivatives thereof; a 1,5-substituted pyrimidine is substituted at the 1-position with a group selected from substituted sugar, unsubstituted sugar, substituted thio-sugar, unsubstituted thio-sugar, substituted carbocyclic, and unsubstituted carbocyclic; a 5-haloalkyl substituted pyrimidine; a 5-bromovinyl substituted pyrmidine; a 5′-phosphoryl derivative of pyrimidine; a 5′-phosphoramidate derivative of pyrimidine; and (E)-5-(2-bromovinyl)-2′-deoxy-5′-uridyl phenyl L-alaninylphosphoramidate.

Suitable nucleoside transport inhibitors include, but are not limited to one or more selected from the group consisting of dipyridamole (DP), p-nitrobenzylthioinosine (NBMPR), 6-benzylaminopurine, 2′,3′-dideoxyguaosine, 8-bromoadenine, 9-[(2-hydroxyethoxy)methyl] guanine (Acyclovir), 9-[(1,3-dihydroxy-2-propoxy) methyl] guanine (Ganciclovir), adenine, hypoxanthine, allopurinol, dilazep, cytochalasin B, lidoflaxine, mioflazine, phloretin, phloridzine, and benzylisoquinoline alkaloids. Suitable benzylisoquinoline alkaloids are selected from the group consisting of papaverine, ethaverine, laudanosine, noscarpine, and berberine.

In one embodiment the composition comprises and effective amount of (E)-5-(2-bromovinyl)-2′-deoxy-5′-uridyl phenyl L-alaninylphosphoramidate and dipyridoamole. In another embodiment, the composition comprises and effective amount of (E)-5-(2-bromovinyl)-2′-deoxy-5′-uridyl phenyl L-alaninylphosphoramidate and p-nitrobenzylthioinosine.

The compositions are useful to inhibit the growth of hyperproliferative cells that express a target enzyme in vitro, in vivo and ex vivo. An effective amount of the composition is delivered to the cells or subject to achieve the desired therapeutic result. Examples of hyperproliferative cells include, but are not limited to, cancer cells such as sarcoma cells, leukemia cells, carcinoma cells, or adenocarcinoma cells. Specific cancers include, but are not limited to, colorectal cancer cells, head and neck cancer cells, breast cancer cells, hepatoma cells, liver cancer cells, pancreatic carcinoma cells, esophageal carcinoma cells, bladder cancer cells, gastrointestinal cancer cells, ovarian cancer cells, skin cancer cells, prostate cancer cells, and gastric cancer cells. The cancer cells can be present in a heterogenous population of cells such as a tumor. In one aspect, the cancer is breast cancer. In another embodiment, the cancer is colon cancer.

In one embodiment, the activity of the target enzyme has been greatly enhanced in the cell as a result of loss of tumor suppressor function and/or selection resulting from previous exposure to chemotherapy, e.g., treatment with 5-FU.

Another aspect of this invention is an assay for screening for novel combinations of therapeutics and ECTA prodrugs. A population of cells that express a target enzyme is contacted with an ECTA prodrug and a candidate agent. The population of cells can be engineered to express the target enzyme or can overexpress the target enzyme in the native environment, i.e., in the subject from which the cells were isolated, e.g., cancer cells several of which are described above. A second population of cells is contacted with the prodrug and test agent; however, the second population of cells is the normal non-hyperproliferative counterpart to pathological cells of the first sample. For example, normal breast cells are the normal counterpart to breast cancer cells. When the cells are engineered, the second population of cells express the target enzyme at “normal” or at least lower levels than the first population of cells. In a further aspect, control populations are assayed concurrently and under the same conditions as the first and second populations. Examples of control populations include normal and hyperproliferative cells that do not receive amounts of the prodrug and candidate agent. A synergistic combination is one that inhibits the growth or kills the cells that express the target enzyme at a high level and at a rate or amount greater than the normal cells receiving the combination. As is apparent to one of skill in the art, various modifications can be made to this assay without departing from the spirit and scope thereof, e.g., varying the concentrations of prodrug and test agent as well as expression level of the target enzyme. Kits to perform such assays containing the reagents and instructions necessary to complete the assay and analyze the results are also provided by this invention.

Further provided is a method for treating or ameliorating the symptoms of disease in a subject suffering from a pathology characterized by the presence of hyperproliferative cells by delivering to the subject a composition containing an effective amount of an ECTA prodrug and nucleoside transport inhibitor. The compositions can be used alone or in combination with other chemotherapeutics or alternative anti-cancer therapies such as radiation. Examples of hyperproliferative cells include, but are not limited to, cancer cells such as sarcoma cells, leukemia cells, carcinoma cells, or adenocarcinoma cells. Specific cancers include, but are not limited to, colorectal cancer cells, head and neck cancer cells, breast cancer cells, hepatoma cells, liver cancer cells, pancreatic carcinoma cells, esophageal carcinoma cells, bladder cancer cells, gastrointestinal cancer cells, ovarian cancer cells, skin cancer cells, prostate cancer cells, and gastric cancer cells. The cancer cells can be present in a heterogenous population of cells such as a tumor.

In one embodiment, the activity of the target enzyme has been greatly enhanced in the cell as a result of loss of tumor suppressor function and/or selection resulting from previous exposure to chemotherapy.

A further aspect of this invention is the preparation of a medicament for use in treating a subject suffering from a pathology characterized by cells expressing a target enzyme.

A still further aspect of this invention is a method for identifying the optimal therapeutic for a subject, by isolating cells expressing a target enzyme and contacting the cells with at least one of the compositions of this invention, and then identifying which of the one or more compositions inhibits the proliferation or kills the cells, thereby identifying the optimal therapeutic.

Yet further provided is a method to enhance the cytotoxicity of an ECTA prodrug against a cell overexpressing an intracellular target enzyme by contacting the cell with the ECTA prodrug and an effective amount of a nucleoside transport inhibitor, as described herein.

### MODES FOR CARRYING OUT THE INVENTION

The practice of the present invention will employ, unless otherwise indicated, conventional techniques of molecular biology, microbiology, cell biology, organic chemistry, medicinal chemistry and recombinant DNA, which are within the skill of the art. See, e.g., Sambrook et al., MOLECULAR CLONING: A LABORATORY MANUAL, 2^{nd} edition (1989); CURRENT PROTOCOLS IN MOLECULAR BIOLOGY, F. M. Ausubel et al. eds., (1987); the series METHODS IN ENZYMOLOGY, Academic Press, Inc.; PCR 2: A PRACTICAL APPROACH, M. J. MacPherson et al., eds. (1995); Spector, D. L. et al. (1998) CELLS: ALABORATORY MANUAL, Vols I to III, Cold Spring Harbor Press; ANIMAL CELL CULTURE, R. I. Freshney, ed. (1987); and J. March, ADVANCED ORGANIC CHEMISTRY: REACTIONS, MECHANISMS AND STRUCTURE, 4^{th} edition (John Wiley & Sons, NY (1992).

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The term “overexpression” shall mean at least 2 fold, preferably 3 fold, more preferably 4 fold and most preferably 5 fold or more expression over normal levels or levels measured from normal or non-pathological cells.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin REMINGTON'S PHARM. SCI., 15th Ed. (Mack Publ. Co., Easton (1975)).

An “effective amount” is an amount sufficient to effect beneficial or desired results. An effective amount can be administered in one or more administrations, applications or dosages.

The term “alkyl” refers to and covers any and all groups which are known as normal alkyl, branched-chain alkyl and cycloalkyl. As used herein, “alkyl” is intended to include both branched and straight-chain saturated aliphatic hydrocarbon groups having the specified number of carbon atoms. Examples of alkyl include, but are not limited to, methyl, ethyl, n-propyl, i-propyl, n-butyl, s-butyl, t-butyl, n-pentyl, and s-pentyl.

“Haloalkyl” is intended to include both branched and straight-chain saturated aliphatic hydrocarbon groups having the specified number of carbon atoms, substituted with 1 or more halogen (for example—CᵥF_{w} where v=1 to 3 and w=1 to (2v+1)). Examples of haloalkyl include, but are not limited to, trifluoromethyl, trichloromethyl, pentafluoroethyl, and pentachloroethyl.

“Cycloalkyl” is intended to include saturated ring groups, such as cyclopropyl, cyclobutyl, or cyclopentyl.

The term “alkenyl” refers to and covers normal alkenyl, branch chain alkenyl and cycloalkenyl groups having one or more sites of unsaturation. Similarly, the term alkynyl refers to and covers normal alkynyl, and branch chain alkynyl groups having one or more triple bonds. “Alkynyl” is intended to include hydrocarbon chains of either a straight or branched configuration and one or more triple carbon-carbon bonds which may occur in any stable point along the chain, such as ethynyl and propynyl.

Lower alkyl means the above-defined broad definition of alkyl groups having 1 to 6 carbons in case of normal lower alkyl, and as applicable 3 to 6 carbons for lower branch chained and cycloalkyl groups. Lower alkenyl is defined similarly having 2 to 6 carbons for normal lower alkenyl groups, and 3 to 6 carbons for branch chained and cyclo-lower alkenyl groups. Lower alkynyl is also defined similarly, having 2 to 6 carbons for normal lower allynyl groups, and 4 to 6 carbons for branch chained lower alkynyl groups.

Some of the compounds of the present invention may have trans and cis (E and Z isomers. In addition, the compounds of the present invention may contain one or more chiral centers and therefore may exist in enantiomeric and diasteromeric forms. Still further oxi and related compounds of the present invention may exist in syn and anti isomeric forms. The scope of the present invention is intended to cover all such isomers per se, as well as mixtures of cis and trans isomers, mixtures of syn and anti isomers, mixtures of diastereomers and racemic mixtures of enantiomers (optical isomers) as well. In the present application when no specific mention is made of the configuration (cis, trans, syn or anti or R or S) of a compound (or of an asymmetric carbon) then a mixture of such isomers, or either one of the isomers is intended. In a similar vein, when in the chemical structural formulas of this application a straight line representing a valence bond is drawn to an as etric carbon, then isomers of both R and S configuration, as well as their mixtures are intended. Defined stereochemistry about an asymmetric carbon is indicated in the formulas (where applicable) by a solid triangle showing beta configuration, or by a hashed line showing alpha configuration.

“Target” or “pathological” cells include hyperproliferative cells that are de-differentiated, immortalized, neoplastic, malignant, metastatic or transformed. Examples include, but are not limited to, cancer cells such as sarcoma cells, leukemia cells, carcinoma cells, or adenocarcinoma cells. Specific cancers include, but are not limited to, colorectal cancer cells, head and neck cancer cells, breast cancer cells, hepatoma cells, liver cancer cells, pancreatic carcinoma cells, esophageal carcinoma cells, bladder cancer cells, gastrointestinal cancer cells, ovarian cancer cells, skin cancer cells, prostate cancer cells, and gastric cancer cells. The cancer cells can be present in a heterogenous population of cells such as a tumor.

Target or pathological cells overexpress an intracellular enzyme that is related to any of a loss of tumor suppressor gene product function, drug resistance or genetic instability. Alternatively, resistance to one drug may confer resistance to other, biochemically distinct drugs. Unlike prior art therapies directed to creating more potent inhibitors of endogenous, intracellular enzymes, ECTA prodrugs exploit the higher enzyme activity associated with therapy-resistant diseased cells and tissues versus normal cells and tissues and do not rely on inhibiting the enzyme. The term “target enzyme” is used herein to define enzymes having one or more of the above noted characteristics.

Gene products activated or overexpressed and related to drug resistance include, but are not limited to thymidylate synthase (TS) (Lönn, U. et al. (1996), Kobayashi, H. et al. (1995), and Jackman, A. L. et al. (1995b)), dihydrofolate reductase (Banerjee, D. et al. (1995) and Bertino, J. R. et al. (1996)), tyrosine kinases (TNF-α) (Hudziak, R. M. et al. (1988)) and multidrug resistance (Stühlinger, M. et al. (1994), Akdas, A. et al. (1996), and Tannock, I. F. (1996)); and ATP-dependent multidrug resistance associated proteins (Simon, S. M. and Schindler, M. (1994)) and, in some diseases including colon and prostate cancer, topoisomerase I (Husain et al. (1994)).

Amplification of dihydrofolate reductase (DHFR) is related to resistance to methotrexate while amplification of the gene encoding thymidylate synthase is related to resistance to tumor treatment with 5-fluoropyrimidine. Amplification of genes associated with drug resistance can be detected and monitored by a modified polymerase chain reaction (PCR) as described in Kashini-Sabet, et al. (1988), U.S. Pat. No. 5,085,983, or the method described herein. Acquired drug resistance can be monitored by the detection of cytogenetic abnormalities, such as homogeneous chromosome staining regions and double minute chromosomes both of which are associated with gene amplification. Alternative assays include direct or indirect enzyme activity assays, each of which are associated with gene amplification (e.g., Carreras, C. W. and Santi, D. V. (1995)) and other methodologies (e.g. polymerase chain reaction, Houze, T. A. et al. (1997) or immunohistochemistry (Johnson, P. G. et al. (1997)).

The enzyme glutathione-S-transferase was shown to be occasionally elevated in some human tumors (Morgan, A. S. et al. (1998)), but nevertheless is excluded from “target enzyme” as used herein because it is a member of a gene family encoding enzymes with overlapping specificities.

Thus, in one aspect, this invention provides compositions comprising an effective therapeutic amount of an ECTA prodrug that is selectively converted to a toxin in the cell by an endogenous, intracellular enzyme (“target enzyme”) and an agent or composition that inhibits nucleoside transport in a cell. Examples of prodrugs that are selectively converted to the toxin in the cell by the target enzyme, include but are not limited to a 1,5-substituted pyrimidine derivative, a 5-substituted pyrimidine derivative wherein the substituent at the 5 position is extractable from the pyrimidine ring by the target enzyme, e.g., an alkyl, an alkenyl, an alkynyl, a vinyl, a propargyl and substituted derivatives thereof. In a further aspect, the 2-substituent is or contains a toxophore.

In another embodiment, the 1,5-substituted pyrimidine derivative is substituted at the 1-position with a group selected from a substituted sugar, an unsubstituted sugar, a substituted thio-sugar, an unsubstituted thio-sugar, a substituted carbocyclic, and an unsubstituted carbocyclic. Examples of such include but are not limited to a 2-haloalkyl substituted pyrimidine, e.g., a 5-bromovinyl substituted pyrimidine.

Further embodiments of the 1,5-substituted pyrimidine derivative is a 5′-phosphoryl derivative of pyrimidine and a 5′-phosphoramidate derivative of pyrimidine.

Suitable nucleoside transport inhibitors include one or more selected from the group consisting of dipyridamole (DP), p-nitrobenzylthioinosine (NBMPR), 6-benzylaminopurine, 2′,3′-dideoxyguanosine, 8-bromoadenine, 9-[(2-hydroxyethoxy)methyl] guanine (Acyclovir), 9-[(1,3-dihydroxy-2-propoxy) methyl] guanine (Ganciclovir), adenine, hypoxanthine, allopurinol, dilazep, cytochalasin B, lidoflaxine, mioflazine, phloretin, phloridzine, and benzylisoquinoline alkaloids. Suitable benzylisoquinoline alkaloids are selected from the group consisting of papaverine, ethaverine, laudanosine, noscarpine, and berberine.

In another aspect, the invention provides a method to enhance the cytotoxity of an ECTA compound against a cell containing a target enzyme by contacting the cell with an effective amount of a nucleoside inhibitor compound. It further provides a methods to inhibit the growth of a cell containing a target enzyme or a hyperproliferative cell by contacting the cell with an effective amount of a composition comprising an ECTA prodrug that is selectively converted to a toxin in the cell by an endogenous, intracellular enzyme and a nucleoside transport inhibitor.

In a still further aspect, the invention provides a method for treating a pathology characterized by hyperproliferative cells in a subject by delivering to the subject an effective amount of a composition comprising an ECTA prodrug that is selectively converted to a toxin in the cell by an endogenous, intracellular enzyme and a nucleoside transport inhibitor.

ECTA prodrugs that have been shown to be activated by target enzymes as defined herein are the L and D isomers of the compounds having one of the following structures:

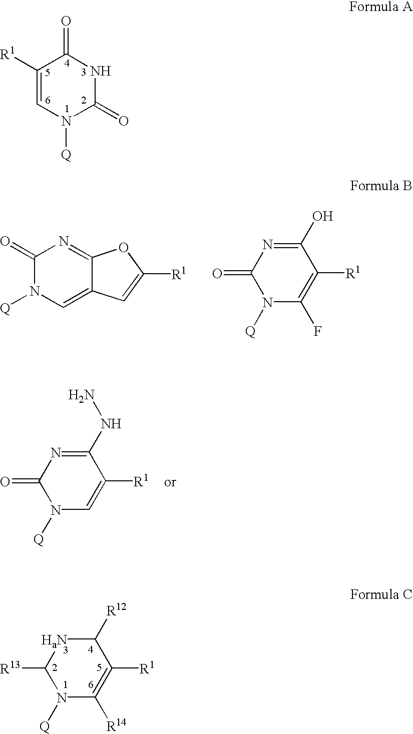

or tautomers thereof, wherein in Formula C, R¹² or R¹³ may be the same or different and are selected from the group consisting of oxo, OH or NHNH₂, wherein a is 0 or 1, providing that if a is 0 and R¹³ is oxo, then a double bond exits between position 3 and 4 and R² is NHNH₂; further providing that if a is 0 and R¹² is oxo, then a double bond exists between position 2 and 3 and R¹³ is NHNH₂; further providing that if a is 1, then R¹² and R¹³ are both oxo.

In the above formulae (A, B and C), R¹ (at the 5-position) is or contains a leaving group which is a chemical entity that has a molecular dimension and electrophilicity compatible with extraction from the pyrimidine ring by an endogenous, intracellular enzyme, and which upon release from the pyriinidine ring by the endogenous, intracellular enzyme, has the ability to inhibit the proliferation of the cell or kill the cell. A preferred embodiment for the substituent in the R¹ position is one that could undergo an allylic interchange.

An example of a leaving group is an alkenyl group of the formula, i.e., (—CH═CH)ₙ—R⁴, wherein n is 0 or an integer from 1 to 10, and R⁴ is a halogen such as is I or Br, CN or mercury, or alternatively, R¹is or contains a group selected from hydrogen, alkyl, alkene, alkyne, hydroxy, —O-alkyl,—O-aryl, —O-heteroaryl, —S-alkyl, —S-aryl, a cyanide, cyanate, thiocyanate halovinyl group, halomercuric group, —S-heteroaryl, —NH₂, —NH-alkyl, —N(alkyl)₂, —NHCHO, —NHOH, —NHO-alkyl, NH₂CONHO—, and NHNH₂. For example, when n is 0 or an integer from 1 to 10, R⁴ is —CH₂-O-A, wherein A is a phosphoramide derivative, or a compound of the formula:

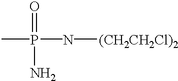

Alternatively, in the above formulae (A, B or C), R¹ is a moiety of the formula:

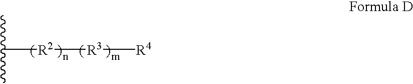

wherein, R⁴ is a toxophore moiety. As used herein, the term “toxophore” shall mean a moiety which is or contains a leaving group which is a chemical entity that has a molecular dimension and electrophilicity compatible with extraction from the pyrimidine ring by an endogenous, intracellular enzyme and which upon release from the pyrimidine ring by the endogenous, intracellular enzyme, has the ability to inhibit the proliferation of the cell or kill the cell.

In one aspect of Formula D, R² is or contains a divalent electron conduit moiety. In one embodiment, R² is or contains a mono- or polyunsaturated electron conduit acting to conduct electrons away from the pyrimidine ring and toward the leaving group R⁴. In one embodiment, R² is selected from the group consisting of an unsaturated hydrocarbyl group, an aromatic hydrocarbyl group comprising one or more unsaturated hydrocarbyl groups, and a heteroaromatic group comprising one or more unsaturated hydrocarbyl groups.

In a yet further aspect, m is 0 and R² is selected from the group consisting of:

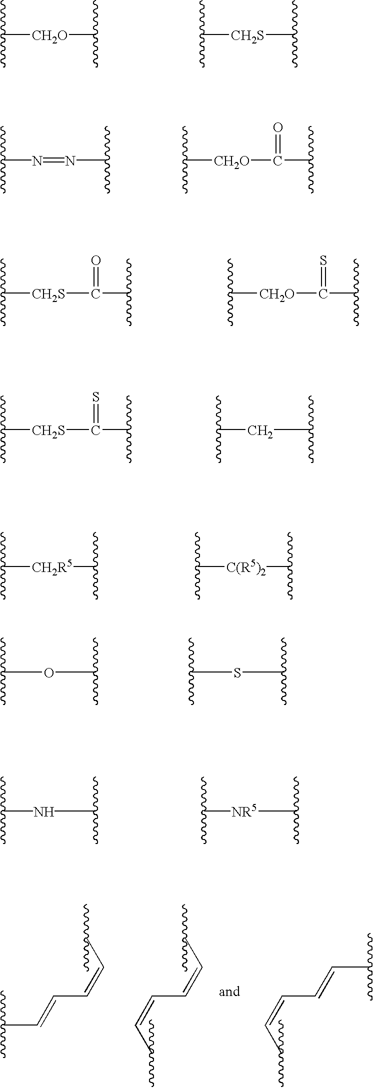

wherein R⁵ is independently the same or different and is selected from the group consisting of a linear or branched alkyl group having from 1 to 10 carbon atoms, a cycloalkyl group having from 3 to 10 carbon atoms, CN and a halogen.

In one embodiment of Formula D, R² is an unsaturated hydrocarbyl group having a structure selected from the group consisting of:

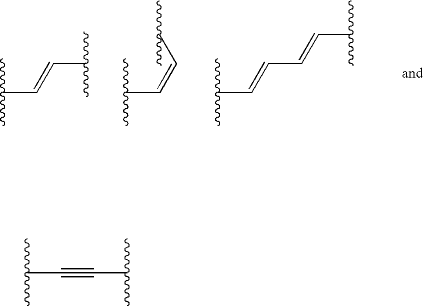

In another embodiment of Formula D, R² is an aromatic hydrocarbyl group having a structure selected from the group consisting of:

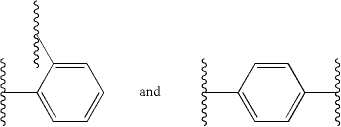

In yet another embodiment of Formula D, R² is a heteroaromatic group having a structure selected from the group consisting of:

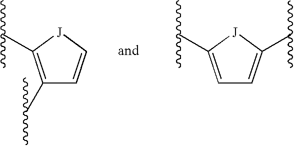

wherein J is a heteroatom, such as —O—, —S—, or —Se', or a heteroatom group, such as —NH— or —NR^{ALK}—, where R^{ALK} is a linear or branched alkyl having 1 to 10 carbon atoms or a cycloalkyl group having 3 to 10 carbon atoms.

In an alternative embodiment of Formula D, R³ is a divalent spacer moiety, also referred to as a spacer unit. Divalent spacers include, but are not limited to, a moiety having a structure:

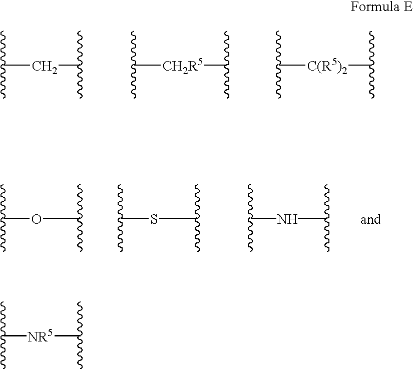

wherein R¹is the same or different and is independently a linear or branched alkyl group having from 1 to 10 carbon atoms, or a cycloalkyl group having from 3 to 10 carbon atoms.

In an alternative aspect of Formula D, R³ is a divalent spacer moiety having a structure selected from the group consisting of:

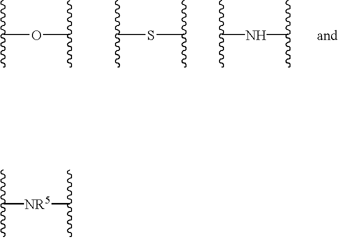

In yet another aspect of Formula D, R² and R³, taken together form a structure selected from the group consisting of:

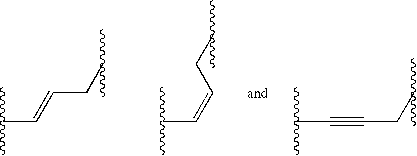

In one embodiment, the toxophore (R⁴ in Formula D or R¹ in Formulae A, B or C) is or contains a leaving group that is activated or released by an intracellular enzyme overexpressed in the cell. In one embodiment, R⁴ is or contains a group having a structure selected from the group consisting of F, Cl, Br, I, CN, SO₃H, CO₂H, CO₂CH₂CH₃, CO₂CH₃, SI(CH₃)₃, CHO, NO₂, CF₃, CCl₃, CH═C(R¹⁵)₂ and a derivative of cisplatin, such as:

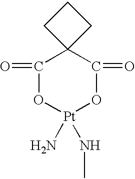

or a substituent selected from the structures:

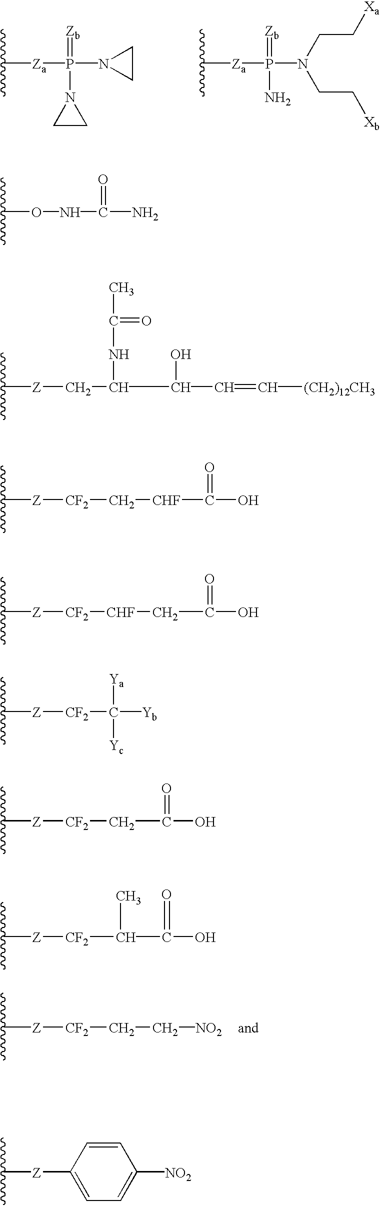

wherein Xₐ and X_{b} are independently the same or different and are selected from the group consisting of Cl, Br, I, and a potent leaving group and wherein Yₐ, Y_{b} or Y_{c} are independently the same or different and are hydrogen or F and wherein Z, Zₐ and Z_{b} are independently the same or different and are selected from the group consisting of O and S; and with respect to Formula C, R¹⁴ is hydrogen or F, providing if R¹⁴ is F, then a is 1 and R¹² and R¹³ are both oxo.

In all of the above noted compounds (Formula A, B and C), Q is a sugar group, a thio-sugar group, a carbocyclic group or an acyclic carbon group as well as 5′-phosphory or phosphoramidate derivatives thereof. Examples of sugar groups include, but are not limited to, monosaccharide cyclic sugar groups such as those derived from oxetanes (4-membered ring sugars), furanoses (5-membered ring sugars), and pyranoses (6-membered ring sugars). Examples of furanoses include threo-furanosyl (from threose, a four-carbon sugar); erythro-furanosyl (from erythrose, a four-carbon sugar); ribo-furanosyl (from ribose, a five-carbon sugar); ara-furanosyl (also often referred to as arabino-furanosyl; from arabinose, a five-carbon sugar); xylo-furanosyl (from xylose, a five-carbon sugar); and lyxo-furanosyl (from lyxose, a five-carbon sugar). Examples of sugar group derivatives include “deoxy”, “keto”, and “dehydro” derivatives as well as substituted derivatives. Examples of thio sugar groups include the sulfur analogs of the above sugar groups, in which the ring oxygen has been replaced with a sulfur atom. Examples of carbocyclic groups include C₄ carbocyclic groups, C₅ carbocyclic groups, and C₆ carbocyclic groups which may further have one or more substituents, such as —OH groups.

In one embodiment, Q is selected from the group consisting of:

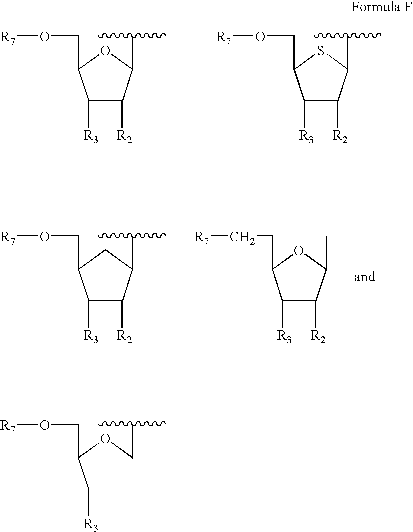

In the above Formula F, R₂ and R₃ are independently the same or different and are selected from the group consisting of Br, Cl, F, I, H, OH, OC(═O)CH₃, —O— and —O—Rg, wherein Rg is a hydroxyl protecting group other than acetyl. R₇ is attached to Q at the 5′ position of Q and is selected from the group consisting of a hydrogen, a hydroxyl, a phosphate group, a phosphodiester group or a phosphoramidate group. R₇ is selected from the group consisting of a hydrogen, a masked phosphate, a phosphoramidate, and derivatives thereof, and wherein R₂ and R₃ are the same or different and are independently hydrogen, —OH —OC(═O)CH₃, or —O—Rg wherein Rg is a hydroxyl protecting group other than acetyl. Any of the members of Formulae F may be in any enantiomeric, diasteriomeric, or stereoisomeric form, including D-form, L-form, α-anomeric form, and β-anomeric form.

In a specific embodiment, Q has the formula:

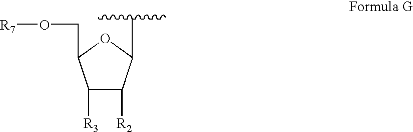

wherein R₂ and R₃ are independently the same or different and are independently H, —OH, —OC(═O)CH₃, or —O—Rg, wherein Rg is a hydroxyl protecting group other than acetyl.

In a further specific embodiment, Q has the following structure:

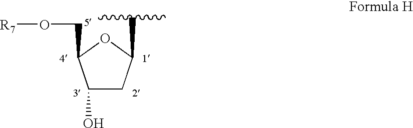

In each of Formulae F, Q or H, R₇ is selected from the group consisting of hydrogen, a masked phosphate or a phosphoramidate and derivatives thereo, and wherein R₂ and R₃ are the same or different and are independently hydrogen or —OH. Alternatively, R₇ is a phosphoramidate group derived from an amino acid, including, for example, the twenty naturally occurring amino acids, e.g., alanine and tryptophane. Examples of such include, but are not limited to:

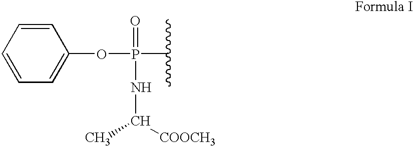

Formula H and its method for preparation, are described in McGuigan et al. (1993), and McGuigan et al. (1996). Additional examples of 5′ substituents are:

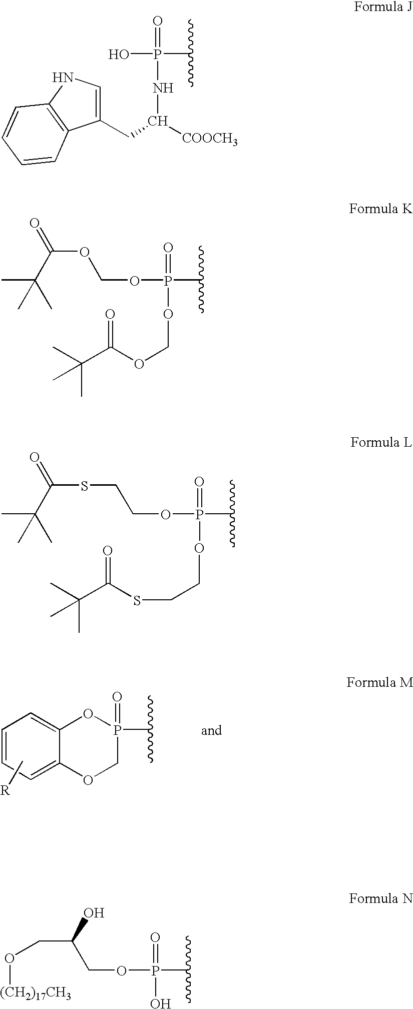

The group identified herein as Formula J, and methods for its preparation, are described in Abraham et al., (1996). Formula K and its method for preparation are described in Freed et al. (1989); Sastry et al., (1992); Farquhar et al. (1994), and Farquhar et al. (1995). Formula L and its method for preparation are described in Valette et al. (1996); and Benzaria et al. (1996). Formula M and its method of preparation are described in Meier et al. (1997); Meier et al., (1997); and Meier et al., (1997). Formula N and its method for preparation, are described in Hostetler et al. (1997); and Hostetler et al., published International Patent Application No. WO 96/40088 (1996).

In one embodiment, the R₇ forms a cyclic group within Q. One such embodiment, and a method for its preparation, is shown below (where DMTr is 4,4′-dimethoxytrityl, Boc is t-butyloxycarbonyl, DCC is 1,3-dicyclohexylcarbodiimide, and 4-DMAP is 4-dimethylaminopyridine):

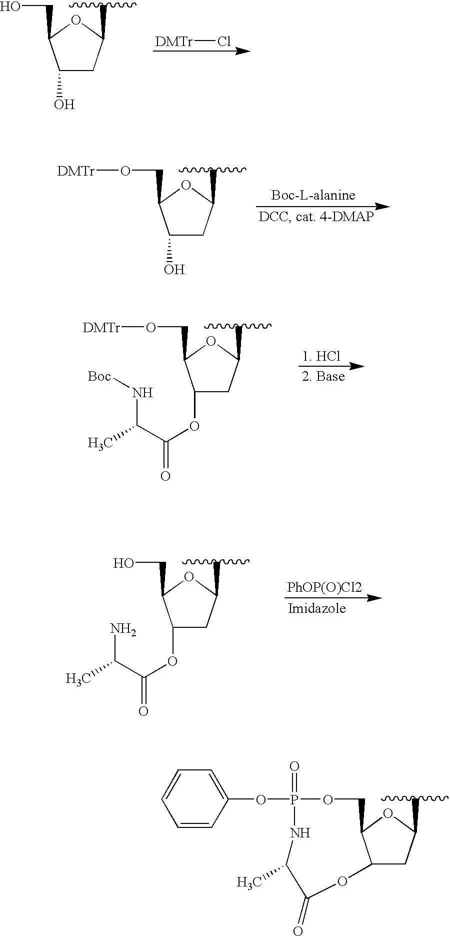

In one embodiment, the ECTA prodrug may be in any enantiomeric, diasteriomeric, or stereoisomeric form, including, D-form, L-form, α-anomeric form, and β-anomeric forms. In an alternative embodiment, the compound may be in a salt form, or in a protected or prodrug form, or a combination thereof, for example, as a salt, an ether, or an ester.

Specific ECTA prodrug compounds having the L or D structures are shown in Table I, below. Compounds are identified by structure and a numerical designation.

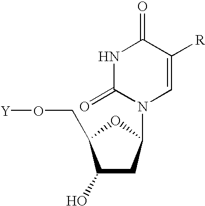

| R | 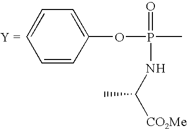 | Y═H |
|---|---|---|
| 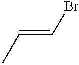 | NB 1011 | NB 1015 (BVdU) |
| 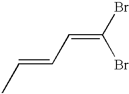 | NB 1012 | — |
| 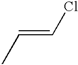 | NB 1013 | NB 1020 |
| —CF₃ | NB 1014 | NB 1027 |
| 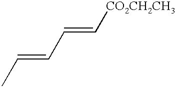 | NB 1016 | NB 1021 |
| 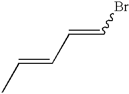 | NB 1017 | NB 1024 |
| 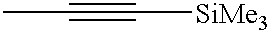 | NB 1018 | NB 1022 |
| 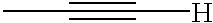 | NB 1019 | NB 1023 |
| 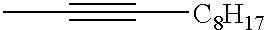 | — | — |
| —C₈H₁₇ | — | — |

More specifically, several ECTA prodrug embodiments are shown below.

A compound having the structure:

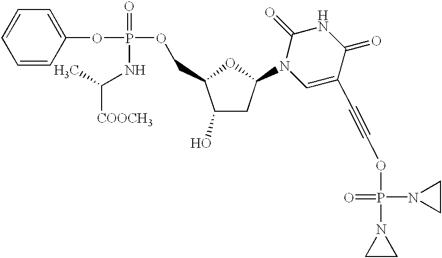

or the nucleoside analog thereof.

A compound having the structure:

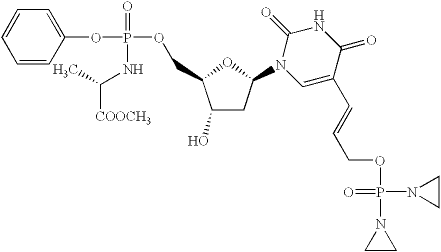

or the nucleoside analog thereof.

A compound having the structure:

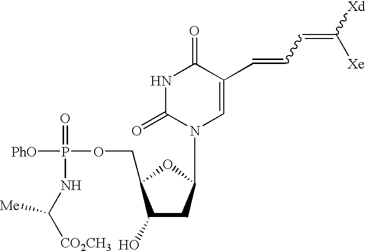

wherein X_{d} and Xₑ are independently the same or different and are selected from the group consisting of Cl, Br, I, and CN or the nucleoside analogs thereof. In a more preferred aspect, X_{d} is Cl or Br and Xₑ is hydrogen.

A compound having the structure:

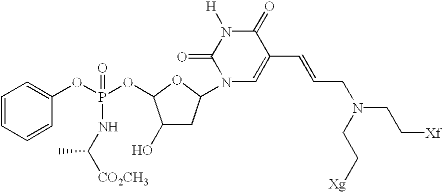

wherein X_{f} and X_{g} are independently the same or different and are selected from the group consisting of Cl, Br, I, and CN, or the nucleoside analogs thereof. In a preferred embodiment, X_{f} and X_{g} are the same and are each is Cl or Br.

A compound having the structure of the formula:

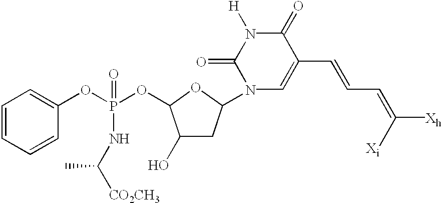

wherein Xₕ and Xᵢ are independently the same or different and are selected from the group consisting of Cl, Br, I, and CN, or the nucleoside analogs thereof. In a preferred embodiment, Xₕ and Xᵢ are independently the same or different and are C or Br and in a more preferred embodiment, Xₕ and Xᵢ are both Br.

A compound having the structure:

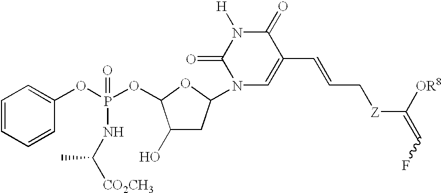

wherein R⁸ is a lower straight or branched chain alkyl, or the nucleoside analogs thereof.

A compound having the structure:

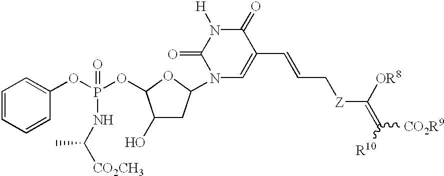

wherein R⁸ and R⁹ are lower straight or branched chain alkyls and R¹⁰ is hydrogen or CH₃, or the nucleoside analogs thereof.

A compound having the structure:

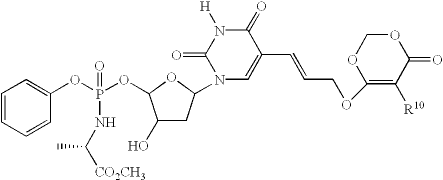

wherein R¹⁰ is hydrogen or CH₃, or the nucleoside analogs thereof.

A compound having the structure:

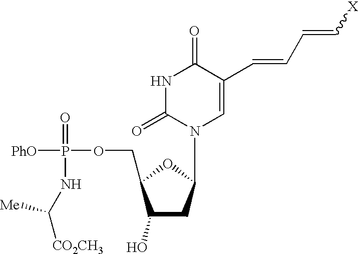

wherein X is selected from the group consisting of CO₂Et, Cl, and Br; or the nucleoside analogs thereof.

In a separate embodiment, the above structures are further modified to possess thiophosphodiaziridine instead of phosphodiaziridine groups, using the methods described below.

The prodrugs can be combined with a carrier, such as a pharmaceutically acceptable carrier, for use in vitro and in vivo. In one embodiment, the ECTA prodrug is in a salt form, or in a protected or prodrug form, or a combination thereof, for example, as a salt, an ether, or an ester.

Salts of the prodrugs of the present invention may be derived from inorganic or organic acids and bases. Examples of acids include hydrochloric, hydrobromic, sulfuric, nitric, perchloric, fumaric, maleic, phosphoric, glycollic, lactic, salicyclic, succinic, toluene-p-sulfonic, tartaric, acetic, citric, methanesulfonic, ethanesulfonic, formic, benzoic, malonic, naphthalene-2-sulfonic and benzenesulfonic acids. Other acids, such as oxalic, while not in themselves pharmaceutically acceptable, can be employed in the preparation of salts useful as intermediates in obtaining the compounds of the invention and their pharmaceutically acceptable acid addition salts. Examples of bases include alkali metal (e.g., sodium) hydroxides, alkaline earth metal (e.g., magnesium) hydroxides, ammonia, and compounds of formula NW₄⁺, wherein W is C₁₋₄ alkyl.

Examples of salts include: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylproprionate, picrate, pivalate, propionate, succinate, tartrate, thiocyanate, tosylate and undecanoate. Other examples of salts include anions of the compounds of the present invention compounded with a suitable cation such as Na⁺, NH₄⁺, and NW₄⁺ (wherein W is a C₁₋₄ alkyl group).

For therapeutic use, salts of the compounds of the present invention will be pharmaceutically acceptable. However, salts of acids and bases which are non-pharmaceutically acceptable may also find use, for example, in the preparation or purification of a pharmaceutically acceptable compound.

Esters of the prodrugs or compounds identified by the method of this invention include carboxylic acid esters (i.e., —O—C(═O)R) obtained by esterification of the 2′-, 3′-and/or 5′-hydroxy groups, in which R is selected from (1) straight or branched chain alkyl (for example, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted by, for example, halogen, C₁₋₄alkyl, or C₁₋₄alkoxy or amino); (2) sulfonate esters, such as alkylsulfonyl (for example, methanesulfonyl) or aralkylsulfonyl; (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di-(C₆₋₂₄)acyl glycerol. In such esters, unless otherwise specified, any alkyl moiety present advantageously contains from 1 to 18 carbon atoms, particularly from 1 to 6 carbon atoms, more particularly from 1 to 4 carbon atoms. Any cycloalkyl moiety present in such esters advantageously contains from 3 to 6 carbon atoms. Any aryl moiety present in such esters advantageously comprises a phenyl group. Examples of lyxo-furanosyl prodrug derivatives of the present invention include, for example, those with chemically protected hydroxyl groups (e.g., with O-acetyl groups), such as 2′-O-acetyl-lyxo-furanosyl; 3′-O-acetyl-lyxo-furanosyl; 5′-O-acetyl-lyxo-furanosyl; 2′,3′-di-O-acetyl-lyxo-furanosyl and 2′,3′,5′-tri-O-acetyl-lyxo-furanosyl.

Ethers of the compounds of the present invention include methyl, ethyl, propyl, butyl, isobutyl, and sec-butyl ethers.

In a further embodiment, the substrate may not be chemically related to pyrimidines or folates, but rather synthesized based upon known parameters of rational drug design. See Dunn, W. J. et al. (1996).

This invention also provides a quick and simple screening assay that will enable initial identification of novel compounds and combinations with at least some of the desired characteristics. The assay requires at least two cell types, the first being a control cell in which the target enzyme is not expressed or is expressed at a low level, e.g., a normal cell. The second cell type is the test cell in which the target enzyme is expressed at a detectable level, e.g., a high level. This cell can be a tumor cell line that is selected for enhanced levels of target enzymes. Alternatively, a cell genetically modified to differentially express the target enzyme or enzymes (containing the appropriate species of target enzyme) can be used. Transfection of host cells with polynucleotides encoding the target enzyme is either transient or permanent using procedures well known in the art and described by Chen, L. et al. (1996), Hudziak, R. M. et al. (1988), or Carter, P. et al. (1992), and in the experimental section below. The cells can be procaryotic (bacterial such as E. coli) or eucaryotic. The cells can be mammalian or non-mammalian cells, e.g., mouse cells, rat cells, human cells, fingi (e.g., yeast) or parasites (e.g., Pneumocystis or Leishmania) which cause disease.

Suitable vectors for insertion of the cDNA are commercially available from Stratagene, La Jolla, Calif. and other vendors. The amount of expression can be regulated by the number of copies of the expression cassette introduced into the cell or by varying promoter usage. The level of expression of enzyme in each transfected cell line can be monitored by immunoblot and enzyme assay in cell lysates, using monoclonal or polyclonal antibody previously raised against the enzyme for immuno-detection. (Chen, L. et al. (1996)). Enzymatic assays to detect the amount of expressed enzyme also can be performed as reviewed by Carreras, C. W. and Santi, D. V. (1995), or the method described in the experimental section below.

In a further aspect, more than one species of target enzyme can be used to separately transduce separate host cells, so that the effect of the candidate drug on a target enzyme can be simultaneously compared to its effect on another enzyme or a corresponding enzyme from another species.

In another embodiment, a third target cell is used as a control because it receives an effective amount of an ECTA prodrug compound of this invention. This embodiment is particularly useful to screen for new agents and combinations of agents that are activated by thymidylate synthase or other ECTA enzymes. In yet a further aspect, at least one additional test cell system is set up to test the synergistic potential of the test therapeutic in combination with a known therapy or agent.

For the purposes of this invention, the successful candidate drug will block the growth or kill the test cell type, but leave the control cell type unharmed. Growth assays can be performed by standard methods as described by Miller, J. H. (1992), Sugarman, B. J. et al. (1985), and Spector, D. L. et al. (1998), or using the methods described in the experimental section below.

The compositions can be directly added to the cell culture media and the target cell or the culture media is then assayed for the amount of label released from the candidate prodrug if the prodrug contains a detectable label. Alternatively, cellular uptake may be enhanced by packaging the prodrug into liposomes using the method described in Lasic, D. D. (1996) or combined with cytofectins as described in Lewis, J. G et al. (1996).

The compositions are useful to predict whether a subject will be suitably treated by this invention by delivering said composition to a sample containing the cell to be treated and assaying for cell death or inhibition of cell proliferation. Applicants provide kits for determining whether a pathological cell or a patient will be suitably treated by this therapy by providing at least one composition of this invention and instructions for use.

This invention also provides a method for inhibiting the proliferation of a pathological or hyperproliferative cell in vitro or in vivo by delivering to the cell an effective amount of a composition of this invention. When practiced in vivo, the method is useful to treat a pathology characterized by hyperproliferative cells in a subject by delivering to the subject an effective amount of a composition of this invention.

When the target hyperproliferative cell is resistant to a chemotherapeutic drug, the method can be further modified by contacting or administering to the cell or patient an effective amount of the drug to which the cell has developed resistance. Because the compositions of this invention can reverse resistance to the prior therapy, subsequent to successful treatment with a composition of this invention, administration of the previous therapy can again inhibit growth or metastasis of tumors. Examples where this may occur include, but are not limited to when the hyperproliferative cell expresses an enzyme that is amplified as a result of selection in vivo by chemotherapy or when the target enzyme is an endogenous intracellular enzyme that is overexpressed in the cell. An example of such an enzyme is thymidylate synthase which has been shown to be overexpressed as a result of prior chemotherapy and confers a drug resistant phenotype on the cell to the prior drug.

The compositions of this invention can also be combined with other known therapies to enhance or synergize the therapeutic effects of either or both prior therapies or the therapeutic effect of the prodrug. Such prior therapies include, but are not limited to cancer chemotherapy, radiation therapy and surgery.

When delivered to an animal (in vivo), the method also is useful to further confirm efficacy of the composition. As an example of an animal model, groups of nude mice (Balb/c NCR nu/nu female, Simonsen, Gilroy, Calif.) are each subcutaneously inoculated with about 10⁵ to about 10⁹ hyperproliferative, cancer or target cells as defined herein. When the tumor is established, the prodrug is administered, for example, by intraperitoneal or intravenous routes. Tumor measurements to determine reduction of tumor size are made in two dimensions using venier calipers twice a week. Other animal models may also be employed as appropriate. (Lovejoy et al. (1997), Clarke, R. (1996), and Pegram, M. D. et al. (1997)).

Administration in vivo can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents can be found below.

The compositions can be used in the manufacture for medicaments for the treatment of humans and other animals by administration in accordance with conventional procedures, such as an active ingredient in pharmaceutical compositions.

The pharmaceutical compositions can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to a composition of the present invention, the pharmaceutical compositions can also contain other pharmaceutically active compounds or a plurality of compounds of the invention.

More particularly, a composition of the formula of the present invention also referred to herein as the active ingredient, may be administered for therapy by any suitable route including oral, rectal, nasal, topical (including transdermal, aerosol, buccal and sublingual), vaginal, parental (including subcutaneous, intramuscular, intravenous and intradermal) and puhnonary. It will also be appreciated that the preferred route will vary with the condition and age of the recipient, and the disease being treated.

Ideally, the composition should be administered to achieve peak concentrations of the active compound at sites of disease. This may be achieved, for example, by the intravenous injection of the composition, optionally in saline, or orally administered, for example, as a tablet, capsule or syrup containing the active ingredient. Desirable blood levels of the composition may be maintained by a continuous infusion to provide a therapeutic amount of the active ingredient within disease tissue. The use of operative combinations is contemplated to provide therapeutic combinations requiring a lower total dosage of each component antiviral agent than may be required when each individual therapeutic compound or drug is used alone, thereby reducing adverse effects.

While it is possible for the composition ingredient to be administered alone, it is preferable to present it as a pharmaceutical formulation comprising at least one active ingredient, as defined above, together with one or more pharmaceutically acceptable carriers therefore and optionally other therapeutic agents. Each carrier must be “acceptable” in the sense of being compatible with the other ingredients of the formulation and not injurious to the patient.

Formulations of the present invention suitable for oral administration may be presented as discrete units such as capsules, cachets or tablets, each containing a predetermined amount of the active ingredient; as a powder or granules; as a solution or suspension in an aqueous or non-aqueous liquid; or as an oil-in-water liquid emulsion or a water-in-oil liquid emulsion. The active ingredient may also be presented a bolus, electuary or paste.

A tablet maybe made by compression or molding, optionally with one or more accessory ingredients. Compressed tablets may be prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) and/or surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin or sucrose and acacia; and mouthwashes comprising the active ingredient in a suitable liquid carrier.

Pharmaceutical compositions for topical administration according to the present invention may be formulated as an ointment, cream, suspension, lotion, powder, solution, past, gel, spray, aerosol or oil. Alternatively, a formulation may comprise a patch or a dressing such as a bandage or adhesive plaster impregnated with active ingredients and optionally one or more excipients or diluents.

For diseases of the eye or other external tissues, e.g., mouth and skin, the formulations are preferably applied as a topical ointment or cream containing the active ingredient in an amount of, for example, about 0.075 to about 20% w/w, preferably about 0.2 to about 25% w/w and most preferably about 0.5 to about 10% w/w. When formulated in an ointment, the composition may be employed with either a paraffinic or a water-miscible ointment base. Alternatively, the ingredients may be formulated in a cream with an oil-in-water cream base.

If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound that enhances absorption or penetration of the ingredients through the skin or other affected areas. Examples of such dermal penetration enhancers include dimethylsulfoxide and related analogues.

The oily phase of the emulsions of this invention may be constituted from known ingredients in a known manner. While this phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier that acts as a stabilizer. It is also preferred to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulfate.

The choice of suitable oils or fats for the formulation is based on achieving the desired cosmetic properties, since the solubility of the active compound in most oils likely to be used in pharmaceutical emulsion formulations is very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the ingredients. The ingredients are preferably present in such formulation in a concentration of about 0.5 to about 20%, advantageously about 0.5 to about 10%, particularly about 1.5% w/w.

Formulations for rectal administration may be presented as a suppository with a suitable base comprising, for example, cocoa butter or a salicylate.

Formulations suitable for vaginal administration may be presented as suppositories, tampons, creams, gels, pastes, foams or spray formulations containing in addition to the ingredients, such carriers as are known in the art to be appropriate.

Formulations suitable for nasal administration, wherein the carrier is a solid, include a coarse powder having a particle size, for example, in the range of about 20 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the ingredients.

Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. The formulations may be presented in unit-dose or multi-dose sealed containers, for example, ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

It should be understood that in addition to the ingredients particularly mentioned above, the formulations of this invention may include other agents conventional in the art having regard to the type of formulation in question, for example, those suitable of oral administration may include such further agents as sweeteners, thickeners and flavoring agents.

Compositions of the formula of the present invention may also be presented for the use in the form of veterinary formulations, which may be prepared by methods that are conventional in the art.

The following examples are intended to illustrate, but not limit, the invention.

MATERIALS AND METHODS

Synthesis of Nucleoside ECTA Compounds

Synthesis of the above noted 5-substituted pyrimidine derivatives can be accomplished by methods that are well-known in the art, for example as described in Applicants' patent literature: PCT/US98/16607 and PCT/US99/01332.

One method requires treatment of 5-chloromercuri-2′-deoxyuridine with haloalkyl compounds, haloacetates or haloalkenes in the presence of Li₂PdCl₄ to form, through an organopalladium intermediate, the 5-alkyl, 5-acetyl or 5-alkene derivative, respectively (Wataya, Y. et al. (1979) and Bergstrom, D. E. et al. (1984)). Another example of C5-modification of pyrimidine nucleosides and nucleotides is the formation of C5-trans-styryl derivatives by treatment of unprotected nucleotide with mercuric acetate followed by addition of styrene or ring-substituted styrenes in the presence of Li₂PdCl₄ (Bigge, et al. (1980)).

For the purpose of this invention, pyrimidine deoxyribonucleoside triphosphates were derivatized with mercury at the 5 position of the pyrimidine ring by treatment with mercuric acetate in acetate buffer at 50° for 3 hours (Dale, et al. (1973)). Such treatment also would be expected to be effective for modification of monophosphates. Alternatively, a modified triphosphate could be converted enzymatically to a modified monophosphate, for example, by controlled treatment with alkaline phosphatase followed by purification of monophosphate. Other moieties, organic or nonorganic, with molecular properties similar to mercury but with preferred pharmacological properties could be substituted. For general methods for synthesis of substituted pyrimidines see, for example, U.S. Pat. Nos. 4,247,544, 4,267,171, and 4,948,882 and Bergstrom, D. E. et al. (1981). The above methods would also be applicable to the synthesis of derivatives of 5-substituted pyrimidine nucleosides and nucleotides containing sugars other than ribose or 2′-deoxyribose, for example 2′-3′-dideoxyribose, arabinose, furanose, lyxose, pentose, hexose, heptose, and pyranose. An example of a 5-position substituent is the halovinyl group, e.g. (E)-5-(2-bromovinyl)-2′-deoxyuridylate (Barr, P. J. et al. (1983)).

Alternatively, 5-bromodeoxyuridine, 5-iododeoxyuridine, and their monophosphate derivatives are available commercially from Glen Research, Sterling, Va. (USA), Sigma-Aldrich Corporation, St. Louis, Mo. (USA), Moravek Biochemicals, Inc., Brea, Calif. (USA), ICN, Costa Mesa, Calif. (USA) and New England Nuclear, Boston, Mass. (USA). Commercially-available 5-bromodeoxyuridine and 5-iododeoxyuridine can be converted to their monophosphates either chemically or enzymatically, through the action of a kinase enzyme using commercial available reagents from Glen Research, Sterling, Va. (USA) and ICN, Costa Mesa, Calif. (USA). These halogen derivatives could be combined with other substituents to create novel and more potent antimetabolites.

The structures at the 5-position of uracil in Formulae A, B and C are referred to as the tethers because they connect the proposed leaving group (toxophore) to the heterocycle. Upon activation of the heterocycle by reaction with the cysteine residue in the active site of a human enzyme, TS, for example, a negative charge is conducted from the 6-position of uracil into the tether. This mechanism has been described for the 5′-monophosphorylated versions of (E)-5-(bromovinyl)-2′-deoxyuridine (BVdU) by Barr, P. J. et al. (1983) and of (E)-5-(3,3,3-trifluoro-1-propenyl)-2′-deoxyuridine (TFPe-dUrd) by Wataya, Y. et al. (1979), Santi, D. V. (1980); and Bergstrom, D. E. et al. (1984).

The tether “spacer” between the toxin and dUMP must be unsaturated so that it can conduct the toxin-labilizing negative charge supplied by the TS-Cys-sulfhydryl attack. Of the many unsaturated organic functionalities available for this purpose, the vinyl, allyl, and propargyl units are simple, small, and readily accessible synthetically. The vinyl and allyl units have the advantage that they can be prepared in either of two non-interconvertible geometric isomeric forms. Thus, they can be used as “probes” of prodrug accommodation by the enzyme active site. On the other hand, the propargyl unit has the advantage of being cylindrically symmetrical, so that enzyme catalyzed toxin release from this type of tether does not depend upon its orientation with respect to dUMP's uracil ring, as is the case with the vinyl and allyl molecules. Alternatively, synthesis based on the structure of BVdU monophosphate and features a leaving group/toxin directly attached to the terminus of a (poly)vinyl substituent at C5 of dUMP. This is the vinyl tether approach. A yet further approach is based on the structure of TFPe-dUMP and is similar to the vinyl tether approach but has a methylene unit separating the leaving group/toxin and the unsaturated unit and thus contains an allyl or propargyl unit. This is the allyl tether approach.

The mechanism of activation of a propargyl version of the allyl tether approach has a precedent in the interaction of both 5-ethynyl-2′-deoxyuridine 5′-monophosphate (EdUMP) and 5-(3-hydroxy-1-propynyl)-2′-deoxyuridine 5′-monophosphate (HOPdUMP) with TS (Barr, P. J. et al. (1981) and Barr, P. J. and Robins, M. J. (1981)). EdUMP is a potent inhibitor of TS (Ki=0.1 TM), and likely forms an allene-based species at the active site. HOPdUMP (Ki=3.0 TM) shows unusual inhibition kinetics, which might be due to formation of a cumulene-based species at the active site.

5-Alkylidenated 5,6-dihydrouracils similar in structure to the intermediate common to both the vinyl and allyl tether approach mechanisms have been synthesized recently (Anglada et al. 1996). These were shown to be highly electrophilic. Their ready reaction with ethanol to generate 5-(ethoxymethyl)uracil is a precedent for the water addition that regenerates catalytically competent TS. Even more recently, the existence of the long-elusive C5 methylene intermediate produced by TS was demonstrated by trapping studies (Barrett, J. E. et al. (1998)).

The compounds of Formula B are defined by the structure of the uracil base, or modified uracil base present. These classes are ECTA compounds where: 1) the base is a furano-pyrimidinone derivative of uracil; 2) the base is 6-fluoro uracil; 3) the base is 4-hydrazone substituted uracil derivative; and 4) the base is uracil. The uracil or modified uracil derived base is used to synthesize compounds substituted with toxic leaving groups at the 5 position, attached by an electron conduit tether at this 5 position, and including an appropriate spacer moiety between the electron conduit and the toxic leaving group. The ECTA compounds can be unphosphorylated, 5′ monophosphate, 5′ phosphodiester, or 5′ protected (“masked”) deoxyuridines or comparable derivatives of alternative carbohydrate moieties, as described below. Protected 5-substituted deoxyuridine monophosphate derivatives are those in which the phosphate moiety has been blocked through the attachment of suitable chemical protecting groups. Protection of 5-substituted deoxyuridine monophosphate derivatives can improve solubility, facilitate cellular penetration, facilitate passage across the blood-brain barrier, and prevent action of cellular or extracellular phosphatases, which might otherwise result in loss of the phosphate group. In another embodiment, 5-substituted uracil or uridine derivatives are administered to cells containing nucleoside kinase activity, wherein the 5-substituted uracil/uridine derivative is converted to a 5-substituted uridine monophosphate derivative. Uridine derivatives may also be modified to increase their solubility, cell penetration, and/or ability to cross the blood-brain barrier.

Synthesis of ECTA Compounds with Propargyl Tethers

The synthesis of propargylic and allylic alcohol-equipped 2′-deoxyuridines is straightforward. Many of these and their close derivatives are reported in the literature, and some have even been studied in connection with TS. For example, 5-alkynyl-dUMPs including the 5-(3-methoxy-1-propynyl) and 5-(3-hydroxy-1-propynyl) ones have been examined as TS inhibitors (Barr, P. J. and Robins, M. J. (1981)) and some of these have been shown to become incorporated into the DNA of TS-deficient cancer cells (Balzarini, J. et al. (1985)).

Both 5-mercuri- (Ruth, J. L. et al. (1978)) and 5-iodouridines (Robins, M. J. et al. (1981)) readily condense with alkenes and alkynes in the presence of a palladium catalyst to afford C5 tether-equipped uridines. The latter route is the more often employed (Robins, M. J. et al. (1982) and Asakura, J. et al. (1988) and (1990)). High-yielding condensations of protected 5-iodo-2′-deoxyuridines with t-butyidimethylsilyl propargyl ether (Graham, D. et al. (1998); De Clercq, E. et al. (1983), methyl propargyl ether (Tolstikov, V. V. et al. (1997)) and even propargyl alcohol itself (Chaudhuri, N. C. et al. (1995) and Goodwin, J. T. et al. (1993)) have been achieved. The 3-hydroxy-1-propynyl substituent introduced by the latter reaction can also be accessed by DIBAL-H reduction of a methacrylate group (Cho, Y. M. et al. (1994)), itself arising from the same Heck reaction used in the synthesis of BVdU. These palladium-catalyzed reactions are so versatile that they can used to condense very long and elaborately-functionalized propargyl-based tethers to 5-iodo-2′-deoxyuridines. (Livak, K. J. et al. (1992) and Hobbs, F. W. Jr. (1989)). (Z)-Allyl-based tethers are generated by the partial hydrogenation of a propargylic precursor over Undiar catalyst (Robins, M. J. et al. (1983)) whereas the (E)-allyl-based ones are best prepared by Heck coupling of an (E)-tributylstannylated ethylene (Crisp, G. T. (1989)).

Closely following the literature procedures, a t-butyldimethylsilyl propargyl ether-equipped 3′,5′-di-O-protected 2′-deoxyuridine (Graham, D. et al. (1998), and De Clercq, E. et al. (1983)) is prepared and a portion of it, converted to the corresponding (Z)-allyl ether, (Robins, M. J. and Barr, P. J. (1983)) is reduced. Because the TBAF-mediated removal of a TBDMS group generates an oxyanion that can be functionalized in situ, these TBDMS-protected propargyl- and (Z)-allytic-tethered nucleosides will serve as convenient precursors to some of the toxophore-equipped targets. For the (E)-allyl alcohol equipped nucleoside, the known O-tetrahydropyranyl ether derivative is prepared by the literature Heck coupling of an (E)-tributylstannylated ethylene (Crisp, G. T. (1989)).

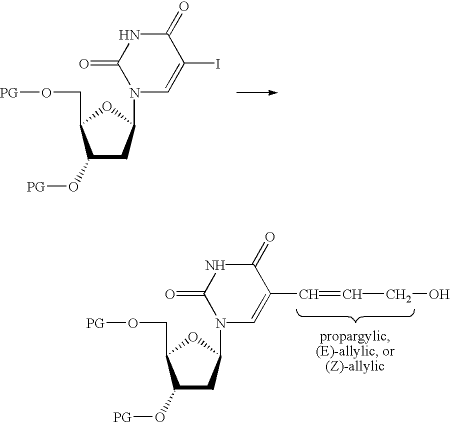

Using a two step literature protocol (Phelps, M. E. et al. (1980) and Hsiao and Bardos (1981)), the propargylic and (E) and (Z)-allylic alcohols are converted to their corresponding bis-aziridinyl phosphoramidates or thiophosphoramidates so that TS processing of the 5′-mononucleotide versions will release an active metabolite of the cytostatic drugs TEPA or ThioTEPA (Dirven, H. A. et al. (1995)), respectively.

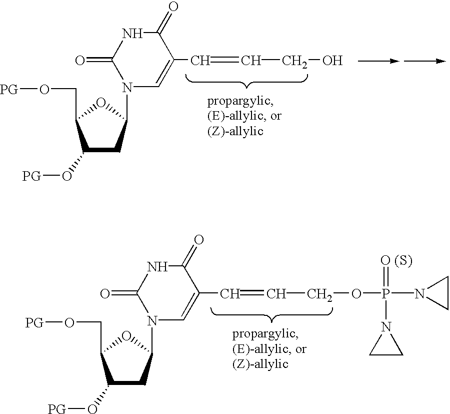

Synthesis of Furano-Pyrimidinones

Synthesis of furano-pyrimidinones begins with synthesis of a C5 propargylic-alcohol-equipped 2′-deoxyuridine. Furano-pyrimidinone compounds are then be formed from the O-tetrahydropyranyl ether derivative described above. Synthesis proceeds by reaction of the second carbon of the propargyl bond with the oxygen attached to the C4 position of the pyrimidine ring to yield a fluorescent furano-pyrimidinone which can be readily separated from the reaction mix. Such compounds provide an additional basis for synthesis of ECTA compounds through various combinations of specific electron conduits, spacers and toxic leaving groups.

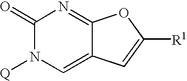

The furo[2,3-d]pyrimidinone nucleosides were prepared by condensing 2′,3′-di-O-p-toluoyl or 2′,3′-di-O-acetyl-5-iodo-2′-deoxyuridine with 1-(tetrahydropyranyloxy)-2-propyne (Jones, R. G. and Mann, M. J. (1953)) under conditions known to promote the formation of these fluorescent compounds (Robins, M. J. et al.(1983)). Base-catalyzed removal of the carbohydrate protecting groups gave the 6-(tetrahydropyran-2-yloxymethyl)-substituted bicyclic nucleoside which was either subjected to standard acidic THP group hydrolysis (TFA in CH₂Cl₂) or was regioselectively 5′-phosphoramidated by the same procedure used to prepare BVdU-PA and 5FUdR-PA. After the phosphoramidation, the THP group could be removed by acidic hydrolysis.

TS ECTA Compounds Based on Furano-Pyrimidinones

Toxic R⁴ leaving groups can be attached to the furan-2 methyl alcohol using methods similar to those employed to attach toxic leaving groups to the hydroxyl on the C5 propargyl uridine compound, as explained with the synthesis of the TEPA and ThioTEPA derivatives described above. A variety of alternative toxic leaving groups, apparent to one skilled in the art, are envisioned. In addition, modifications to the length and composition of the R² electron conduit component and of the composition of the R³ spacer element are also envisioned.

TS ECTA compounds based on furano-pyrimidinones can also consist of variously modified “Q” moieties. Many 5-substituted 2′-deoxyuridines are not substrates for human TK, but interestingly 5-(4-hydroxy-1-butynyl)-2′-deoxyuridine was found to be an exception (Barr, P. J. et al. (1981)). The ECTA compounds can have a free 5′ hydroxyl, a 5′ monophosphate, or a 5′ phosphoramidate group attached to alternative carbohydrate groups. A novel method for synthesis of such phosphoramidate compounds is accomplished by reacting a 2-deoxy 3′-hydroxy, 5′-hydroxy unprotected nucleotide with a phosphochloridate in the presence of an HCl scavenger. In a preferred embodiment, the phosphochloridate comprises a phosphorus substituent which is derived from an amino acid such as alanine. For example, the phosphochloridate can be phenyl-L-methoxyalanine phosphorochloridate.

C6 Fluoro Uridine and C4 Hydozone Based Compounds

The neutral thiol addition to the pyrimidine C5-C6 double bond proceeds as an exothermic reaction (3-9 kcal per mol; see review by Les, A. et al. (1998)) in the normal TS reaction with dUMP. Alternative substituents to the TS reactive hydrogen at the 6 position that can facilitate the formation of the sulfydryl bond with the enzyme, via the active human TS cysteine (homologous with cys-198 of L. casei), include fluorine. Such substituents at other positions in the pyrimidine ring can also facilitate the reaction between the substrate and TS. For instance, a 4-hydrazone substitution on the uracil (as described by Les, A. et al. (1998) facilitates formation of the thiol with TS. It is important that the resulting nucleotide-thiol (TS) intermediate rearranges in such a way as to release the altered nucleotide which can be accomplished passively via hydrolysis.

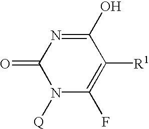

The introduction of fluorine at the C6 position has not been previously reported, but it can be synthesized by following the synthetic descriptions of Krajewskas and Shugar (1982), who describe the synthesis of a number of 6 substituted uracil and uridine analogues.

Chemistry facilitating substitutions at the C4 position of the pyrimidine base are well known by those skilled in the art. Examples of literature descriptions include Wallis et al. (1999); Negishi, et al. (1996), Barbato et al. (1991), Barbato, et al. (1989) and Holy et al. (1999). These synthetic techniques also enable combinations of substitutions, for instance at the C4 and C5 positions of the pyrimidine ring (Pluta, et al. 1999) or the C2 and C4 positions of the pyrimidine ring (Zeid, et al. (1999)).

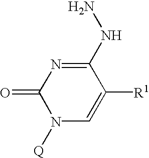

In another embodiment of the invention, ECTA compounds are synthesized by addition of alternative electron conduits, spacer moieties and toxic leaving groups to either the C6 fluoro-uridine base or the C4 hydrazone modified pyrimidine. Methods described above for synthesis of 2, deoxyuridine based ECTA compounds can again be employed for the synthesis of such molecules.

Synthesis of Nucleoside Phenyl Methoxyalaninyl Phosphoramidates

The use of phosphoramidates as phosphate prodrugs for nucleotides was first reported by McGuigan, C. et al. (1993) and McGuigan, C. et al. (1994). These authors showed that phosphoramidate derivatives of antiviral 2′,3′-dideoxynucleoside derivatives such as d4T retain their antiviral activities in thymidine-kinase deficient cells. Further studies showed that the phosphoramidate group was hydrolyzed to the phosphate group inside cells (McGuigan, C. et al. (1996), Balzarini, J. et al. (1996) and Saboulard, et al. (1999)). The phospharamidates were synthesized by reacting 2′,3′-dideoxynucleosides with phenyl methoxyalaninyl phosphorochloridate (PMPC).

Since only one hydroxyl group is present, these reactions usually proceed smoothly. In compounds where more than one hydroxyl group is present, the appropriately protected nucleoside might be required. Since the 5′—OH group of 2′-deoxynucleosides is much less hindered than the 3′—OH group, selective phosphoramidation with PMPC is possible under carefully controlled conditions. Both BVdU and 5FUdR condensed with PMPC in the presence of N-methylimidazole in anhydrous CH₂Cl₂ to give the corresponding phosphoramidates. In both cases, the desired product was readily separable from the starting material using column chromatography on silica gel. The synthetic scheme is summarized below.

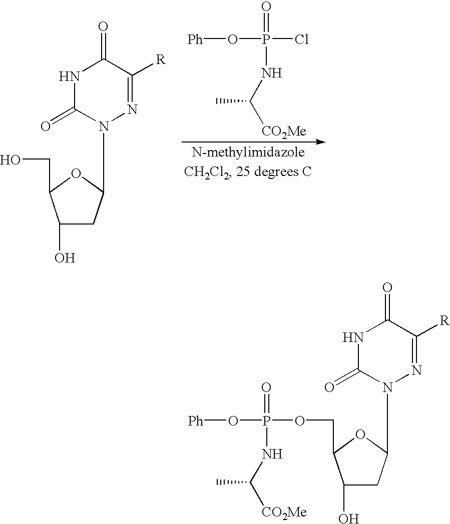

The following examples are intended to illustrate, but not limit the invention.

### EXAMPLES 1 and 2

### Synthesis of ECTA Compounds with Propargyl Tethers

Using the general synthetic procedure described supra, bis-aziridin-1-yl-phosphinic acid 3-[2-deoxyuridin-5-yl]-prop-2-ynyl ester was synthesized and analyzed by ¹H NMR to yield the following result: ¹H NMR ((CD₃)₂SO). Salient features: δ 8.28 (d, 1, H6), 6.10 (pseudo-t, 1, H1′), 5.26 (m, exchanges with D₂O, 1,3′—OH), 5.13 (m, exchanges with D₂O, 1,5′—OH), 4.81 (q or dd, 2, propargyl-CH₂), 4.24 (m, 1, H3′), 3.57 (m, 2,5′-CH₂), 2.15-2.0 (m, 8, aziridine-CH₂).

Bis-aziridin-1-yl-phosphinothioic acid 3-[2-deoxyuridin-5-yl]-prop-2-ynyl ester was also synthesized and analyzed by ¹H NMR to yield the following result: ¹H NMR ((CD₃)₂SO). Salient features: δ 8.29 (d, 1, H6), 6.10 (pseudo-t, 1, H1′), 5.22 (m, exchanges with D₂O, 1,3′—OH), 5.10 (m, exchanges with D₂O, 1,5′—OH), 4.88 (q or dd, 2, propargyl-CH₂), 4.31 (m, 1, H3′), 3.52 (m, 2,5′—CH₂), 2.15-2.0 (m, 8, aziridine-CH₂).

### EXAMPLES 3 to 8

### Synthesis of Furano-Pyrimidinones

Using the general synthetic procedure described supra, the following compounds were prepared.

### EXAMPLE 3

3-(2-Deoxy-β-D-ribofuranosyl)-6-(tetrahydropyran-2-yloxymethyl)furo[2,3-d]pyrimidin-2(3H)-one.

¹H NMR ((CD₃)₂SO) δ 8.80 (s, 1, H4), 6.74 (s, 1, H5), 6.16 (pseudo-t, 1, H1′), 5.27 (d, exchanges with D₂O, 1,3′—OH), 5.12 (t, exchanges with D₂O, 1,5′—OH), 4.72 (m, 1, THP-H2), 4.56 (q, 2, CH₂OTHP), 3.92 (m, 1, H4′), 3.64 (m, 2,5′—CH₂), 2.40 (m, 1, H2′a), 2.03 (m, 1, H2′b), 1.68 and 1.50 (m, 8, THP). Low-resolution mass spectrum (DCI-NH₃) on bis-TMS derivative, m/z 323 (B+TMS+H⁺), 511 (MH⁺), 583 (M+TMS⁺).

### Example 4

3-(2-Deoxy-β-D-ribofuranosyl)-6-(hydroxymethyl)furo [2,3-d]pyrimidin-2(3H)-one.

¹H NMR ((CD₃)₂SO) δ 12.0 (bs, 1, OH), 8.24 (s, 1, H4), 6.53 (s, 1, H5), 5.51 (pseudo-t, 1, H¹′), 4.42 (m, 2, CH₂OH). Low-resolution mass spectrum (DCI-NH₃), m/z 167 (B+2H⁺), 184 (B+NH₄⁺).

### EXAMPLE 5

1-[6-(Tetrahydropyran-2-yloxymethyl)furo[2,3-d]pyrimidin-2(3H)-on-3-yl]-2-deoxy-β-D-ribofuranos-5-yl phenyl methoxy-L-alaninylphosphoramidate.

¹HNMR ((CD3)2SO) complicated due to presence of diastereomers. Salient features: δ 8.62 and 8.59 (each s, each 1, H4), 7.4-7.1 (m, 5, PhO), 6.61 and 6.60 (each s, each 1, H5), 6.25 (m, 1, H1′), 4.56 (q, 2, propargyl-CH₂), 3.56 and 3.54 (each s, each 3, CO₂Me), 2.0 (m, 1, H2′b), 1.22 (m, 3, alaninyl-α-Me). Low-resolution mass spectrum (DCI-NH₃), m/z 167 (B+2H⁺), 184 (B+H⁺+NH₄⁺-THP).

### EXAMPLE 6

1-[6-(Hydroxymethyl)furo[2,3-d]pyrimidin-2(3H)-on-3-yl]-2-deoxy-β-D-ribofuranos-5-yl phenyl methoxy-L-alaninylphosphoramidate.

¹H NMR (CDCl₃) complicated due to presence of diastereomers. Salient features: δ 8.5 (s, 1, H4), 7.4-7.1 (m, 5, PhO), 6.36 and 6.30 (each s, each 1, H5), 6.23 (m, 1, H1′), 3.67 and 3.65 (each s, each 3, CO₂Me), 2.69 (m, 1, H2′a), 2.10 (m, 1, H2′b), 1.35 (m, 3, alaninyl-α-Me). Low-resolution mass spectrum (DCI-NH₃), m/z 525 (MH⁺), 595 (MNH₄⁺).

### EXAMPLE 7

The 4-nitrophenyl ether derivative of 5-(3-hydroxy-1-propynyl)-2′-deoxyuridine was prepared according to standard ether synthesis as shown below.

### EXAMPLE 8

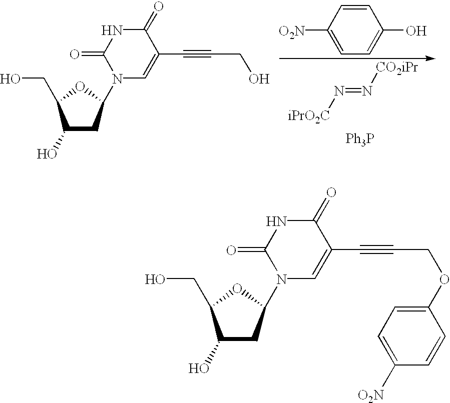

5-[3-(4-Nitrophenoxy)-1-propynyl]-2′-deoxyuridine.

A solution of pre-dried 5-(3-hydroxy-1-propynyl)-2′-deoxyuridine (Robins, M. J. et al. (1983)) (565 mg, 2 mmol) in 40 mL of anhydrous THF under argon was treated with 4-nitrophenol (696 mg, 5 mmol), triphenylphosphine (787 mg, 3 mmol), and diisopropyl azodicarboxylate (590 liters, 3 mmol), and the reaction mixture heated at 60° C. until the solution was clear, and then 1 hour longer. The mixture was allowed to cool to 23° C. and then it was evaporated onto SiO₂ and purified by chromatography using MeOH/CH₂Cl₂ as eluent to afford 107 mg (13%) of the desired ether product: melting point 112-118° C. ¹H NMR ((CD₃)₂SO) 67 11.65 (s, exchanges with D₂O, 1, NH), 8.29 (s, 1, H6), 8.24 (d, J=9.3 Hz, 2, m-ArH), 7.23 (d, J=9.3 Hz, 2, o-ArH), 6.09 (pseudo-t, 1, H1′), 5.17 (s, 2, propargyl-CH₂), 4.22 (m, 1, H3′), 3.80 (m, 1, H4′), 3.59 (m, 2,5′—CH₂), 2.13 (pseudo-t, 2,2′—CH₂). Low-resolution mass spectrum (DCI-NH₃) on per-trimethylsilyated material, m/z 547 [M(TMS)₂H⁺], 565 [M(TMS)₂NH₄⁺], 620 [M(TMS)₃H⁺].

### EXAMPLE 9

### 5-(4-Carbethoxy-1,3-butadienyl)-2′-dexoyuridine

(a) 5-(Carbomethoxyvinyl)-2′-deoxyuridine-3′,5′-bis(tetrahydro-2H-pyran-2-yl)ether (I)

A slurry of 5-(carbomethoxyvinyl)-2′-deoxyuridine (3.0 g, 9.6 mmol), 3,4-dihydro-2H-pyran (22 mL, 21.3 mmol) and pyridinium p-toluenesulfonate (PPTS, 0.242 g, 0.96 mmol) in dimethylformamide (DMF, 5 mL) was stirred at 50° C. for 18 hours. The resulting solution was concentrated in vacuo (bath temperature 45° C.) to give a thick, pale yellow oil. The oil was dissolved in EtOAc and the solid was filtered. The solution was again concentrated. The oil obtained was purified by column chromatography on silica gel using 50-75% EtOAc/hexane as eluent to give 3.81 g (85%) of pure product as a colorless oil.

(b) 5-(3-Hydroxyprop-1-enyl)-2′-deoxyuridine-3′,5′-bis(tetrahydro-2H-pyran-2-yl)ether (II)

A solution of (I) (3.5 g, 7.27 mmol) in CH₂Cl₂ (14 mL) was cooled to −78° C. in a dry ice/acetone bath. Diisobutylaluminum hydride (DIBAL-H) in toluene (1.0 M, 24 mL, 24.0 mmol) was added dropwise over 2 hours while the temperature was maintained at −78° C. The solution was stirred at −78° C. for an additional 2 hours and MeOH (2.5 mL) was added dropwise to destroy any excess DIBAL-H. The reaction mixture was cannulated into a mixture of 30% citric acid solution (50 mL), ice (25 g) and EtOAc (30 mL) over ca. 20 minutes. The phases were separated and the aqueous phase was extracted with EtOAc (2×25 mL). The combined organic phase was washed with saturated NaHCO₃ (20 mL) and brine (20 mL), dried over MgSO₄ and concentrated to give 3.288 g (100%) of colorless oil

(c) 5-(3-Oxoprop-1-enyl)-2′-dexoyuridine-3′,5′-bis(tetrahydro-2H-pyran-2-yl)ether (III)

To a solution of crude (II) obtained from above (1.988 g, 4.4 mmol) in CH₂Cl₂ (9 mL) was added solid pyridinium dichromate (PDC; 1.82 g, 4.8 mmol) with water cooling. The suspension was stirred while acetic acid (0.4 mL) was added dropwise. The water bath was removed and the reaction was stirred at room temperature for 1 hour. The crude product was filtered through a pad of florisil (2×2.5 cm) and the florisil washed with 35 mL EtOAc. The brown solution obtained was filtered through another column of florisil (3.5 cm diam×2.5 cm height). The filtrate was concentrated to give 1.273 g (64% yield) of very light brown oil.

(d) 5-(4-Carbethoxy-1,3-butadienyl)-2′-dexoyuridine-3′,5′-bis(tetrahydro-2H-pyran-2-yl)ether (IV)

(Carbethoxymethylene)triphenylphosphorane (0.32 mg, 0.92 mmol) was added to a solution of the crude aldehyde (III) (0.344 g, 0.77 mmol). The solution darkened and turned rust color. After 1 hour, (III) was completely consumed as judged by thin layer chromatography. The solvent was evaporated and the crude product was purified by column chromatography on silica gel using 35-45% EtOAc/hexane as eluent. The pure product (0.310 g, 78% yield) was obtained as colorless oil.

(e) 5-(4-Carbethoxy-1,3-butadienyl)-2′-dexoyuridine (V)

5-(4-Carbethoxy-1,3-butadienyl)-2′-dexoyuridine-3′,5′-bis(tetrahydro-2H-pyran-2-yl)ether (IV) (0.637 g, 1.22 mmol) was dissolved in MeOH (1.5 mL) and PPTS (0.049 g, 0.16 mmol) was added. The solution was stirred at 50° C. for 7.5 hours and left at room temperature overnight. A white precipitate was formed. The reaction mixture was cooled to 0° C. and filtered to give pure (V) as a white solid (0.188 g). The filtrate was concentrated and chromatographed on silica gel using 50-100% EtOAc/hexane as eluent to give a further 0.180 g product. The total yield of the product was 0.368 g (86%).

¹H NMR (DMSO-d₆): 1.22 (3H, t, J=7 Hz), 2.17 (2H, br t, J=5.5 Hz), 3.55-3.75 (2H, m), 3.81 (1H, m), 4.12 (2H, q, J=7 Hz), 4.25-4.28 (1H, m), 5.19 (1H, t, J=4.8 Hz), 5.27 (1H, d, J=4.1 Hz), 5.98 (1H, d, J=14.5 Hz), 6.14 (1H, t, J=6.3 Hz), 6.75 (1H, d, J=14.5 Hz), 7.18-7.30 (2H, m), 8.30 (1H, s), 11.56 (1H, s).

### EXAMPLE 10

### 5-(4-Carbomethoxy-1,3-butadienyl)-2′-dexoyuridine (Va)

A solution of triethylamine (3.9 mL, 28.2 mmol) in dioxane (12 mL) was deareated by bubbling nitrogen through for 15 minutes. Palladium acetate (0.60 g, 0.26 mmol) and triphenylphosphine (0.183 g, 0.70 mmol) were added and the solution was heated at 70° C. for 20 minutes to give a dark brown solution. 5-Iodo-3′-deoxyuridine (5.0 g, 14.1 mmol) and methyl 2,4-pentadienoate (2.5 g, 22.3 mmol) were added and the mixture was heated under reflux for 15 hours. The solvent and volatile components were evaporated in vacuo and the residue was partitioned between water (15 mL) and EtOAc (15 mL). The phases were separated and the aqueous phase was extracted twice with EtOAc (10 mL each). The combined organic phase was washed with brine and concentrated. The residue was dissolved in MeOH (15 mL) and allowed to cool to room temperature. The solid formed was collected by filtration, washed with a small quantity of MeOH and dried in vacuo to give 0.38 g brown powder.

¹H NMR (DMSO-d₆): 2.17 (2H, t, J=6.4 Hz), 3.55-3.70 (2H, m), 3.66 (3H, s), 3.82 (1H, q, J=3.6 Hz), 4.27 (1H, m), 5.18 (1H, t, J=4.9 Hz), 5.26 (1H, d, J=4.5 Hz), 5.99 (1H, d, J=14.4 Hz), 6.14 (1H, d, J=6.4 Hz), 6.74 (1H, d, J=14.8 Hz), 7.20-7.35 (2H, m), 8.30 (1H, s), 11.56 (1H, s).

The filtrate from above was concentrated and chromatographed on silica gel using 60-100% EtOAc/hexanes as eluent to give another 0.70 g of product as a brown foam. The combined yield was 1.08 g (22.6%).

### EXAMPLE 11

### 5-(4-Carboxy-1,3-butadienyl)-2′-dexoyuridine (VI)

### Method I

5-(4-Carbethoxy-1,3-butadienyl)-2′-dexoyuridine (V, from Example 9) (0.449 g, 1.28 mmol) was dissolved in 2N NaOH (3 mL) and stirred at 25° C. After 20 minutes, a precipitate was formed and TLC showed that the starting material was completely consumed. The mixture was cooled to 0° C. and acidified to pH 1 with 2N HCl. The resulting off-white solid was filtered off, washed with water and dried in vacuo to give 0.225 g (54%) product.

¹H NMR (DMSO-d₆): 2.12-2.19 (2H, m), 3.50-3.70 (2H, m), 3.75-3.85 (1H, m), 4.24-4.29 (1H, m), 5.19 (1H, t, J=4.8 Hz), 5.27 (1H, d, J=4.2 Hz), 5.80-5.95 (1H, m), 6.14 (1H, t, J=6.4 Hz), 6.60-6.75 (1H, m), 7.15-7.25 (2H, m), 8.26 (1H, s), 11.56 (1H, s), 12.16 (1H, br s).

The filtrate and washings were combined and evaporated to dryness. The resulting sticky yellow solid was dissolved in MeOH from which a white precipitate was formed. The solid was filtered off to give an additional 0.200 g of product.

### Method II

The title compound can also be prepared from 5-(4-carbomethoxy-1,3-butadienyl)-2′-dexoyuridine (Va, from Example 10) in comparable yield as mentioned above.

### EXAMPLE 12

### 5-(4-Bromo-1E,3E-butadienyl)-2′-dexoyuridine (VIla) and 5-(4-Bromo-1E,3Z-butadienyl)-2′-dexoyuridine (VIIb)

To a solution of 5-(4-carboxy-1,3-butadienyl)-2′-dexoyuridine (VI) (0.200 g, 0.62 mmol) in DMF (1 mL) was added KHCO₃ (0.185 g, 1.84 mmol) and the mixture was stirred for 20 minutes at 25° C. A solution of N-bromosuccinimide (0.117 g, 0.65 mmol) in DMF (0.3 mL) was added dropwise. Smooth gas evolution (CO₂) occurred throughout the addition. The resulting brown suspension was stirred for 2 hours at 25° C. at which time TLC showed that (VI) was completely consumed. Water (10 mL) was added to the suspension and the resulting solution was extracted with EtOAc (2×15 mL). The extract was dried over MgSO₄ and the solvent was evaporated in vacuo to give a yellow solid (178 mg, 80% yield) consisting of a mixture of two isomers as shown by ¹H NMR. The crude product was separated by semi-preparative HPLC (reversed phase C18 column) using 20% acetonitrile in water as the mobile phase to give the following isomers:

5-(4-Bromo-1E,3Z-butadienyl)-2′-dexoyuridine: retention time 10.5 minutes; ¹H NMR: (DMSO-d₆): 2.11-2.18 (2H, m), 3.50-3.70 (2H, m), 3.80 (1H, distorted q, J=3.5 Hz), 4.25 (1H, br s), 5.08 (1H, br s), 5.25 (1H, br s), 6.15 (1H, t, J=6.5 Hz), 6.40 (1H, d, J=7 Hz), 6.53 (1H, d, J=15.6 Hz), 6.83 (1H, dd, J=7,10 Hz), 7.39 (1H, dd, J=10, 15.6 Hz).

5-(4-Bromo-1E,3E-butadienyl)-2′-dexoyuridine: retention time 15.1 minutes; ¹H NMR (DMSO-d₆): 2.12-2.16 (2H, m), 3.50-3.70 (2H, m), 3.80 (1H, q, J=3.2 Hz), 4.26 (1H, m), 5.13 (1H, br s), 5.25 (1H, br s), 6.14 (1H, t, J=6.5 Hz), 6.36 (1H, d, J=15.6 Hz), 6.67 (1H, d, J=13.1 Hz), 6.84 (1H, dd, J=11, 13.1 Hz), 7.04 (1H, dd, J=11, 15.6 Hz).

### EXAMPLE 13

Using the procedures mentioned in Example 11, Method II, the following compounds can be obtained in a similar fashion: 5-(4-chloro-1,3-butadienyl)-2′-dexoyuridine (using N-chlorosuccinimide in place of N-bromosuccinimide in Step B); 5-(4-iodo-1,3-butadienyl)-2′-dexoyuridine (using iodine in sodium idodide in place of N-bromosuccinimide).

### EXAMPLE 14

Phenyl N-methoxy-L-alaninyl Phosphorochloridate

L-alanine methyl ester hydrochloride (245.8 g; 1.76 mol) was placed in a 12 liter three-neck round bottom flask (equipped with a mechanical stirrer and thermometer) followed by 4.0 liters of dichloromethane. The mixture was stirred for 15 minutes at room temperature. Phenyl phosphodichloridate (370.0 g; 1.76 mol) was added to the mixture and stirring was continued for 15 minutes at room temperature. The flask was placed in the bath with dry ice and the stirring was continued for 20 minutes until a uniform suspension was formed.

Freshly distilled tri-n-butylamine (626.5 g; 3.38 mol) was added dropwise (˜90 minutes) with vigorous stirring to the reaction mixture so that the temperature inside the flask was held at ˜0° C. The bath was removed and the stirring was continued for 6 hours at room temperature. The solution was concentrated to ˜2.84 liters by evaporating several portions of the mixture on a rotary evaporator and the mixture was sealed under argon and stored at −20° C. The product was 85% pure by phosphorus NMR to give an estimated concentration of phenylmethoxyalaninyl phosphochloridate of ˜0.5 M.

### EXAMPLE 15

5-(2-Bromovinyl)-2′-deoxyuridine phenyl N-methoxy-L-alaninyl phosphoramidate (NB1011)

The reaction was performed under argon atmosphere. 5-(2-bromovinyl)-2′-deoxyuridine (BVdU) (204 g; 612 mmol) was placed in three-neck 3 liter round bottom flask equipped with mechanical stirrer. The flask was placed in ice-water bath and 1600 mL (˜800 mmol) of phenylmethoxyalaninyl phosphochloridate reagent were added using an addition funnel over 15 minutes with vigorous stirring of the reaction mixture, followed by the addition of 100 mL of N-methylimidazole over 5 minutes using syringe. After 5 minutes the mixture became clear and after 10 minutes the ice-water bath was removed to allow the mixture to warm up to room temperature while stirring was continued. The reaction was monitored by reversed phase HPLC and was complete in 3 hours. The reaction was quenched by the addition of 100 mL of methanol and the mixture was evaporated to an oil, re-dissolved in 6 liters of dichloromethane and passed through 800 g of silica gel. The major portion of BVdU-PA, referred to herein as NB1011, was passed through the column during the loading and finally the elution of NB1011 was completed by passing 5 liters of 5% methanol in dichloromethane. All fractions containing NB 1011 were combined and evaporated to an oil, the residue was dissolved in 4 liters of ethyl acetate and the mixture was extracted with water (2×2 liters). The organic layer was dried with sodium sulfate, filtered, and washed with ethyl acetate (3×300 mL). The combined filtrate and washings were evaporated to produce a lightly colored white foam; total weight ˜540 g.

The crude product was purified by two silica gel chromatography using 0-5% MeOH in CH₂Cl₂ and 10% MeOH in CH₂Cl₂, respectively, as eluent. The yield of product (>98% pure) was 64 g.

### EXAMPLE 16

Using the methods described in Example 15, the phenyl N-methoxy-L-alanyl phosphoramidates of the following nucleosides were prepared:

1. 5-(4,4-dibromo-1,3-butadienyl)-2′-deoxyuridine;

2. 5-(2-chlorovinyl)-2′-deoxyuridine;

3. 5-trifluoromethyl-2′-deoxyuridine;

4. 5-(4-carbethoxy-1,3-butadienyl)-2′-deoxyuridine;

5. 5-(4-carbomethoxy-1,3-butadienyl)-2′-dexoyuridine;

6. 5-(4-bromo-1E,3E-butadienyl)-2′-deoxyuridine;

7. 5-(4-bromo-1E,3Z-butadienyl)-2′-deoxyuridine;

8. 5-(trimethylsilylethynyl)-2′-deoxyuridine;

9. 5-(ethynyl)-2′-deoxyuridine;

10. 5-(1-decynyl)-2′-deoxyuridine;

11. 3-(2′-deoxy-β-D-ribofuranosyl)-2,3-dihydrofuro[2,3-d]pyrimidin-2-one; and

12. 3-(2′-deoxy-β-D-ribofuranosyl)-6-octyl-2,3-dihydrofuro[2,3-d] pyrimidin-2-one.

Chemical assays for products, for example, where a reaction product is an anti-metabolite of the bromovinyl-derivatives of dUMP, are described in the Examples provided below or by Barr, P. J. et al. (1983).

### EXAMPLE 17

### Materials and Methods

### Biological Assays

Cell lines:

Normal human colon epithelial cells (CCD18co) and skin fibroblasts (Det551) were purchased from ATCC (Rockville, Md.). MCF7TDX, human breast carcinoma cells resistant to 2 μM Tomudex were obtained from Dr. Patrick Johnston, Queens University, Belfast. H630R10, human colorectal carcinoma cells resistant to 10 μM 5-Fluorouracil were obtained from Dr. Edward Chu (Yale Cancer Center) and Dr. Dennis Slamon (UCLA). The MCF7TDX and the H630R10 cell lines have been previously described in Drake, J. C. et al., 1996 and Copur, S. et al., 1995, respectively.

Chemicals:

Dipyridamole and nitrobenzylthioinosine were purchased from ICN Biomedicals (Aurora, Ohio). 5-Fluorouracil was purchased from Sigma (St. Louis, Mo.). Tomudex was provided by Zeneca (Wilmington, Del.).

Culture Conditions:

All cells were cultured under standard conditions of 37° C., 95% humidified air, 5% CO₂ in RPMI 1640 culture medium containing 10% fetal calf serum (Life Technologies) and penicillin/streptomycin/fungizone. MCF7TDX cells were maintained continuously in 2 μM Tomudex, and H630R10 cells were maintained continuously in 10 μM 5-FU. The medium was renewed or the cells were passaged about every three days to maintain optimal growth conditions. Normal cells were passaged a maximum of 15 times to avoid senescence.

Cytotoxicity Studies

384- Well Interaction Screening Assay.

500 cells per well were transferred to a 384-well tissue culture plate (Corning Inc., Corning, N.Y.) and allowed to attach for 24 hours in standard culture conditions. Compounds were then applied in a bidirectional (checker board) pattern (Chou, T. C. and Talalay, P. 1984). Following a 5-day incubation, the redox indicator dye, alamarBlue (AccuMed International, Westlake, Ohio) was added to each well at a 10% v/v ratio, and fluorescence was monitored at 535 excitation, 595 emission. Cytotoxic effect levels and drug interactions were assessed by the combination index method (Chou, T. C. and Talalay, P. 1984 and Bible, K. C. et al. 1997), described briefly below.

96-Well Combination Cytotoxicity Assay.

Exponentially rowing cells were transferred at a density of 1.0−5.5×10³ cells per well to a 96- well tissue culture plate and allowed to attach for 24 hours. Compounds were then applied in duplicate half log serial dilutions. Each compound was tested separately, and mixed together at a single molar ratio approximately equal to the ratio of the individual IC₅₀ values. After an additional 72 hour incubation, cells were washed once with PBS and stained with 0.5% crystal violet in methanol. Plates were washed gently in water to remove unbound stain and allowed to dry overnight. Crystal violet stain bound to the total protein of attached cells was redissolved in Sorenson's buffer (0.025 M sodium citrate, 0.025 M citric acid in 50% ethanol), and absorbance monitored at 535 nM. Sigmoid curves were fit according to the Hill inhibitory Emax model, and IC₅₀ calculated as the average of three or more separate determinations. Where applicable, the combination index for multiple drug effects was calculated according to the median-effect principle (Chou, T. C. and Talalay, P. 1984) using the CalcuSyn software from Biosoft (Ferguson, Mo.). Briefly, the IC₅₀ and the slope parameter (m) for each agent alone were determined from the median effect plot, an x,y plot of log(D) vs log (fₐ/fᵤ) based on Chou's median effect equation:

fₐ/fᵤ=(D/Dₘ)^{m} [Equation 1]

where D=dose of the drug, Dₘ=IC₅₀ as determined from the x-intercept of the median effect plot, fₐ=fraction of cells affected, fᵤ=fraction of cells unaffected (fᵤ =1fₐ), and m=an exponent signifying the steepness of the sigmoid dose-effect curve. Only experiments with linear correlation coefficients (r)>0.9 were accepted for analysis. A combination index (CI) was then calculated to assess synergism or antagonism according to the following equation which assumes an independent mechanism of drug action (mutual exclusivity):

CI=(D)₁/(Dₓ)₁+(D)₂/(Dₓ)₂+(D)₁(D)₂/(Dₓ)₁(Dₓ)₂ [Equation 2]

where (D)₁ and (D)₂ are the concentrations of drug 1 and drug 2 which combined produce x% inhibition, and (Dₓ)₁ and(Dₓ)₂ are the concentrations of each drug which alone produce x% inhibition. CI=1 indicates an additive interaction, CI<1 indicates synergy, and CI>1 indicates antagonism. For each experiment CI's from several different effect levels and concentrations of a constant molar ratio were averaged. Student t-tests were applied to determine if the average differed significantly from 1.

Results:

384-well Screening Studies.

To identify drugs which potentially synergize with NB1011, combination cytotoxicity experiments were performed with NB 1011 and each of 10 antitumor agents from several different mechanistic classes using MCF7TDX and H630R10 tumor cells. Results from these initial 384-well alamarBlue screening assays are shown in Table 2. In general, a combination index of <1 indicates synergy, ˜1 indicates additivity, and >1 indicates antagonism (Pegram, M. D. et al. (1999)).

TABLE 2
Drugs screened for interaction with NB1011

| | | Combination Index ± s.e.m. | |
|---|---|---|---|
| Drug | Class | MCF7TDX | H630R10 |
| Irinotecan | Inhibition of topoisomerase I | 1.36 ± 0.38 | 1.26 ± 0.20 |
| Topotecan | 2.45 ± 0.85 | ND | |
| Etoposide | Inhibition of topoisomerase II | 3.13 ± 0.58 | 1.96 ± 0.28 |
| Vinblastine | Inhibition of microtubule | 1.09 ± 0.16 | 0.78 ± 0.32 |
| | assembly | | |
| Taxol | Stabilization of microtubules | 1.41 ± 0.32 | 0.99 ± 0.15 |
| Cisplatin | DNA damage | 1.51 ± 0.35 | ND |
| Thiotepa | Alkylation | 2.23 ± 0.45 | ND |
| Doxorubicin | Inhibition of nucleic acid | 0.55 ± 0.06 | 1.05 ± 0.13 |
| | synthesis | | |
| 5-fluoroura- | Inhibition of TS, DNA/RNA | 3.19 ± 0.35 | ND |
| cil | incorporation | | |
| Methotrexate | Antifolate, inhibition of | 1.78 ± 0.44 | ND |
| | DHFR, TS | | |

ND=not determined. Combination Index (CI)=1 indicates additivity, CI<1 indicates synergy, and CI>1 indicates antagonism. CI calculated as the average of at least 4 consecutive dose/effect levels. Class of drugs as indicated by Dorr, R. T. and Van Hoff, D. D. (1994).

Two of the ten agents screened, vinblastine and doxorubicin, showed potential synergy (CI≦1.1) with NB1011 in MCF7TDX and H630R10 cell. Two of the remaining 8 agents, irinotecan and taxol showed an additive or antagonistic interaction (CI=1-1.4) with NB1011, while all the other agents showed antagonism (CI>1.5). The most antagonistic interaction was observed with 5-Fluorouracil which gave CI=3.19 in MCF7TDX cells. In light of these results, vinblastine and doxorubicin were chosen for further study using a 96-well crystal violet combination cytotoxicity assay.

96-Well Combination Cytotoxicity Studies.

The 96-well format was chosen for more detailed drug interaction studies. Three additional agents were included in the 96-well assay: oxaliplatin, a new platinum analog DNA damaging agent; dipyridamole (DP) and p-nitrobenzylthioinosine (NBMPR), both potent inhibitors of equilibrative nucleoside transport processes (Belt, J. A. et al. (1993)). Oxaliplatin was tested to confirm the antagonism results for cisplatin. The nucleoside transport inhibitors were tested because published data (Tsavaris, N. et al.(1990), Grem, J. L. (1992) and Wright, A. M. et al. (2000)) suggested they may modulate the activity of nucleoside based drugs. To analyze whether any of these drugs would enhance the activity of NB1011 specifically in tumor cells, two normal cell types, Det551 and CCD18co, were included in the assays. Results of these experiments are shown in Table 3.

TABLE 3
Average combination index (CI) values for drugs tested in combination
with NB1011 in tumor and normal cells

| | | | ± | P | Molar | NB1011 | Drug Dose | Inter- |
|---|---|---|---|---|---|---|---|---|
| Drug | Cell Line | CI | SEM | value | Ratio^{a} | Dose (μM) | (μM) | action^{b} |
| Dipyridamole | H630R10 | 0.75 | 0.11 | 0.052 | 2 | 11-150 | 5.5-75 | Syn |
| | MCF7TDX | 0.51 | 0.06 | 0.001 | 0.2 | 1.1-3.2 | 5.5-16 | Syn |
| | Det551 | 1.17 | 0.23 | 0.484 | 5 | 5.8-375 | 1.2-75 | Add |
| | CCD18co | 1.30 | 0.08 | 0.008 | 5 | 81-375 | 16-75 | Ant |
| p-Nitrobenzyl- | H630R10 | 0.35 | 0.07 | 0.001 | 1 | 1.5-500 | 1.5-500 | Syn |
| Thioinosine | MCF7TDX | 0.57 | 0.17 | 0.029 | 3.33 | 0.15-150 | 0.045-45 | Syn |
| (NBMPR) | Det551 | 1.43 | 0.16 | 0.026 | 3.33 | 32-300 | 9.7-90 | Ant |
| | CCD18co | 3.93 | 1.00 | 0.019 | 3.33 | 32-300 | 9.7-90 | Ant |
| Vinblastine | H630R10 | 0.63 | 0.10 | 0.003 | 6000 | 4.1-54 | 0.0005- | Syn |
| | | | | | | | 0.015 | |
| | MCF7TDX | 1.44 | 0.29 | 0.186 | 2000 | 0.4-1.9 | 0.0005- | Ant |
| | | | | | | | 0.015 | |
| | Det551 | 0.54 | 0.10 | 0.003 | 50000 | 2.9-47 | 0.0005- | Syn |
| | | | | | | | 0.015 | |
| | CCD18co | 0.65 | 0.10 | 0.008 | 50000 | 17-135 | 0.0005- | Syn |
| | | | | | | | 0.015 | |
| Oxaliplatin | H630R10 | 1.78 | 0.06 | 0.001 | 120 | 6.9-150 | 0.1-1.3 | Ant |
| | MCF7TDX | 2.24 | 0.33 | 0.004 | 12 | 0.6-15 | 0.1-1.3 | Ant |
| Doxorubicin | H630R10 | 1.39 | 0.13 | 0.012 | 300 | 117-150 | 0.039-0.5 | Ant |
| | MCF7TDX | 1.96 | 0.25 | 0.004 | 600 | 1.9-15 | 0.001-0.025 | Ant |

^{a}Molar ratio of NB1011:Drug. ^{b}Syn = synergy, Ant = antagonism, Add = additivity.

As can be seen in Table 3, doxorubicin, although promising in the initial screening assay, failed to synergize in the more detailed 96 well cytotoxicity assay (CI=1.39 and 1.96 in H630R10 and MCF7TDX cells, respectively). Oxaliplatin had an antagonistic interaction in the tumor cells (CI=1.78 and 2.24, respectively). Since both oxaliplatin and doxorubicin antagonized NB 1011 in the tumor cells, they were not tested in the normal cell assays. Consistent with the initial screening data, vinblastine synergized with NB1011 in H630R10 cells (CI=0.63), however it antagonized NB1011 in MCF7TDX cells (CI=1.44). Furthermore, in Det551 and CCD18co normal cells, vinblastine interacted synergistically with NB 1011 to a similar extent as in H630R10 cells (CI=0.54 and 0.65, respectively). This lack of selectivity in the potentiation of NB1011 by vinblastine would most likely limit the use of this combination in the clinic. The nucleoside transport inhibitor, dipyridamole, synergized with NB1011 in the tumor cells (CI=0.75 and 0.51), but failed to synergize with NB1011 in the normal cells (CI=1.17 and 1.30). Similarly, NBMPR, another NT inhibitor, showed synergy with NB1011 in the tumor cells (CI=0.35 and 0.57), but produced no synergy in the normal cells (CI=1.43 and 3.93). Taken together this data indicate that 2 of the 13 agents tested, DP and NBMPR, which are both inhibitors of equilibrative nucleoside transport, potentiate the activity of NB1011. This enhancement of NB1011 activity by DP and NBMPR appears specific for the tumor cells tested, since no synergy was observed for these combinations in the two normal cell types analyzed.

While the invention has been described in detail and with reference to specific embodiments thereof, it will be apparent to one skilled in the art that various changes and modifications can be made therein without departing from the spirit and scope thereof.

### REFERENCES

Literature

Abraham et al. (1996) J. Med. Chem. 39: 4569-4575

Akdas, A. et al. (1996) Eur. Urol. 29(4):483-486

Almasan, A. et al. (1995a) Proc. Natl. Acad. Sci. USA 92:5436-5440

Almasan, A. et al. (1995b) Cancer Metastases Rev. 14:59-73

Anglada et al. (1996) J. Heterocyclic Hem. 33:1259-1270.

Antelman, D. et al. (1995) Oncogene 10:697

Asakura, J. et al. (1988) Tetrahedron Lett. 29:2855-2858

Asakura, J. et al. (1990) J. Org. Chem. 55:4928-4933

Aschele, C. et al. (1999) J. Chem. Oncol. 17(6):1760-1770

Balzarini, J. et al. (1985) Methods Find. Exp. Clin. Pharmacol. 7:19-28

Balzarini, J. et al. (1996) Proc. Natl. Acad. Sci. USA 93:7295-7299

Banerjee, D. et al. (1995) Acta Biochem Pol. 42:457

Banerjee, D. et al. (1998) Cancer Res. 58:4292-4296

Barbour, K. W. et al. (1992) Molec. Pharmacol. 42:242-248

Barbato, et al. (1989) Nucleosides Nucleotides 8(4):515-528

Barr, P. J. and Robins, M. J. (1981) J. Med. Chem. 24(12):1385-1388

Barr, P. J. et al. (1983) Biochemistry 22:1696-1703

Barrett, J. E. (1998) J. Am. Chem. Soc. 120:449-450

Belt, J. A. et al. (1993) Adv. Enzyme Regul. 33:235-52.

Benzaria et al. (1996) J. Med. Chem. 39: 4958

Bergstrom, D. E. et al. (1984) J. Med. Chem. 27:279-284

Bergstrom, D. E. et al. (1981) J. Org. Chem. 46(7):1432-1441

Bertino, J. R. et al. (1996) Stem Cells 14:5

Bible, K. C., et al. (1997) Cancer Res. 57(16):3375-80

Bigge, et al (1980) J. Amer. Chem. Soc. 102:2033-2038

Callahan, A. P. et al. (1989) Comm. Nucl. Med. 20:3-6

Carreras, C. W. and Santi, D. V. (1995) Annu. Rev Biochem. 64:721-62.

Carter, P. et al. (1992) Proc. Natl. Acad. Sci. USA 89:4285-4289

Chadhuri, N. C. et al. (1995) J. Am. Chem. Soc. 117:10434-10442

Chen, L. et al. (1996) Cancer Res. 56:1331-1340

Cho, Y. M. et al. (1994) Tetrahedron Lett. 25:1149-1152

Chou, T. C. and Talalay, P. (1984) Adv. Enzyme Regul. 22:27-55

Clarke, R. (1996) Breast Cancer Res. Treat. 39:1-6

Cobleigh, M. A. et al. (1999) J. Clin. Oncol. 17(9):2639-2648

Connors, T. A. and Knox, R. J. (1995) Stem Cells 13:501-511

Copur, S. et al. (1995) Biochem Pharmacol. 49(10):1419-1426

Crisp, G. T. (1989) Synth. Commun. 19:2117-2123

Cruickshank, K. A. et al. (1988) Tetrahedron Lett. 29:5221-5224

Curtin, N. J. et al. (1991) Cancer Res. 51(9):2346-52

Dale, et al. (1973) Proc. Natl. Acad. Sci. USA 70:2238-2242

Dearing, M. P. et al. (1996) J. Cell Biochem Suppl. 24:165-72

DeClercq, E. et al. (1983) J. Med. Chem. 26:661-666

DeClercq, E. et al. (1997) Clin. Micro. Review 10(4):674-693

Dicken, A. P. et al. (1993) Proc. Natl. Acad. Sci. USA 90:11797-11801

Dirven, H. A. et al. (1995) Cancer Res. 55:1701-1706

Dorr, R. T. and Von Hoff, D. D., eds. (1994) “Cancer Chemotherapy Handbook” 2nd ed. publ. by Appleton and Lange), pp. 768-773

Drake, J. C. et al. (1996) Biochem Pharmacol. 51(10):1349-55

Dunn, W. J. et al. (1996) J. Med. Chem. 39:4825-4832

Dyer, R. L. et al. (1991) Nucl. Acids Chem. 4:79-83

Edler, D. et al. (2000) Clin. Cancer Res. 6(2):488-492

Fan, J. and Bertino, J. (1987) Oncogene 14:1191-1200

Farquhar, J. et al. (1994) J. Med. Chem. 37:3902-3909

Farquhar, J. et al. (1995) J. Med. Chem. 38:488-495

Freed, et al. (1989) Biochem. Pharmacol. 38:3193-3198

Freemantle, S. J. et al. (1995) Br. J Cancer 71:925-930

Fries, K. M., et al. (1995) J. Med. Chem. 38:2672-2680

Funk, J. O. (1999) Anticancer Res. 19(6A):4772-4780

Goel, R. et al. (1989) J. Clin. Oncol. 7(2):262-9.

Goodwin, J. T. et al. (1993) Tetrahedron Lett. 34:5549-5552

Gorlick, R. and Bertino, J. R. (1999) Semin Oncol. 26(6):606-11.

Gottesman, M. M. et al. (1995) Annu. Rev. Genet. 29:607

Graham, D. et al. (1998) J. Chem. Soc. Perkin Trans. 1: 1131-1138

Grem, J. L. (1992) Semin Oncol. April 19(2 Suppl 3):56-65.

Griffith, D. A. et al. (1990) Biochem. Pharmacol. 40:2297-2303.

Haskell, C. M. ed., (1995) Cancer Treatment 4th Ed., W. B. Saunders Co., Philadelphia, Pa.

Hobbs, F. W. Jr. (1989) J. Org. Chem. 54:3420-3422

Holy, et al. (1999) J. Med. Chem. 42(12):2064-2086

Hostetler, et al. (1997) Biochem. Pharmacol. 53:1815

Houze, T. A. (1997) Tumour Biol. 18:53-68

Howell, S. B. et al. (1989) Cancer Res. 49(12):3178-83.

Hsiao and Bardos (1981) J. Med. Chem. 24:887-889

Hudziak, R. M. et al. (1988) Proc. Natl. Acad. Sci. USA 85:5102

Husain, et al. (1994) Cancer Res. 54:539

Jackman, A. L. et al. (1995a) Ann. Oncol. 6(9):871-881

Jackman, A. L. et al. (1995b) Anticancer Drug Des. 10:573

Johnson, P. G et al. (1997) J. Clin. Oncol. 15:1923-1931

Jones, R. G. and Mann, M. J. (1953) J. Am. Cancer Soc. 75: 4048-4052

Kashani-Sabet et al. (1988) Cancer Res. 48:5775-5778

Kobayashi, H. et al. (1995) Japan. J Cancer Res. 86:1014-1018

Krajewskas and Snugar (1982) Biochem. Pharmacol. 31(6):1097-1102

Kraup, M. (1995) Gen. Pharmac. 26(6):1185-90.

Lackey, D. B. (2000) Biochemical Pharmacology vol.61

Lasic, D. D. (1996) Nature 380:561-562

Lee, V. et al. (1997) Exp. Cell Res. 234:270-276

Lehman, N. L. (2000) Cancer Chemother Pharmacol. 45(2):142-8

Leichman, C. G. et al. (1997) J. Clin. Oncol. 15(10):3223-9

Lenz, H. J. et al. (1998) Clin. Cancer Res. 4:1227-1234

Lenz, H. J. et al. (1998) Clin. Cancer Res. 4(5):1243-50

Les, A. et al. (1988) Bio. Structure and Dynamics 15(4): 703-715

Lewis, J. G et al. (1996) Proc. Natl. Acad. Sci. USA 93:3176-3181

Lin, W -Y. et al. (1997) Eur J. Nucl. Med. 24:590-595

Livak, K. J. et al. (1992) Nucleic Acids Res. 20:4831-4837

Lönn, U. et al. (1996) Cancer 77(1):107-112

Look, K. Y. et al. (1997) Anticancer Res. 17:2353-2356

Lovejoy, et al. (1997) J. Pathol. 181:130-135

Madec, A. et al. (1988) Bull Cancer 75:187-194

Mader, R. M. et al. (1998) Gen. Pharmacol. 31(5):661-666

Mahony, C. (1982) Clin. Pharmacol Ther. 31(3):330-8

McGuigan, C. (1993) J. Med. Chem. 36:1048-1052

McGuigan, C. (1996) J. Med. Chem. 39:1748-1753

McGuigan, C. et al. (1994) FEBS Lett. 351:11-14

Mclntee, E. J. (1997) J. Med. Chem. 40:3323-3331

Melton, R. G. and Sherwood, R. E. (1996) J. Natl. Cancer Inst. 88:153-165

Meier, et al. (1997a) Bioorg. Med. Chem. Lett. 7:1577

Meier, et al. (1997b) Bioorg. Med. Chem. Lett. 7:99

Meier, et al., (1997c) International Antiviral News. 5:183

Miller, J. H., (1992) “A short course in bacterial genetics: A laboratory manual and handbook for E. coli and related bacteria” Cold Spring Harbor Press

Morgan, A. S. et al. (1998) Cancer Res. 58:2568-2575

Negishi, et al. (1996) Nuc. Acids Symp. Ser 35:137-138

Nelson, J. A. and Drake, S. (1984) Cancer Res. 44(6):2493-6.

Paradiso, A. et al. (2000) Br. J. Cancer 82(3):560-567

Pederson-Lane, J. (1997) Protein Expression and Purification 10:256-262

Pegram, M. D. et al. (1997) Oncogene 15:537-547

Pegram, M. D. et al. (1999) Oncogene. 18(13):2241-51

Phelps, M. E. et al. (1980) J. Med. Chem. 23:1229-1232

Pluta, et al. (1999) Boll. Chim. Farm. 138(1):30-33

Ramu, N. and Ramu, A. (1989) Int. J. Cancer 43(3):487-91

Robins, M. J. et al. (1981) Tetrahedron Lett. 22:421-424

Robins, M. J. et al. (1982) Can. J. Chem. 60:554-557

Robins, M. J. et al. (1983) J. Org. Chem. 48:1854-1862

Romain, S. et al. (1995) Intl. J. Cancer 61(1):7-12

Rooney, P. H. et al. (1998) Cancer Res. 58(22):5042-5

Roth, J. A. et al. (1999) Oncology 13(10 Supp. 5):148-154

Ruth, J. L. et al. (1978) J. Org. Chem. 43:2870-2876

Saboulard, L. et al. (1999) Mol. Pharm. 56:693-704

Santi, D. V. (1980) J. Med. Chem. 23:103-111

Sastry, et al., (1992) Mol. Pharmacol 41:441-445

Schaechter, M. et al., eds. (1993) Mechanisms of Microbial Disease, 2nd Ed., Williams and Wilkins

Schmoll, H. J. (1994) Ann. Oncol. Suppl 3:115-21

Shepard, H. M. et al. (1988) J. Clin. Immunol. 8:353-395

Simon, S. M. and Schindler, M. (1994) Proc. Natl. Acad. Sci. USA 91:3497

Smith, K. A. et al. (1995) Philos. Trans. R. Soc. Lon. B. Biol. Sci. 347:49-56

Spector, D. L. et al. (1998) “Cells, A Laboratory Manual, Vols I to III,” Cold Spring Harbor Press

Stühlinger, M. et al. (1994) J. Steroid Biochem. 49:39

Sugarman, B. J. et al. (1985) Science 230:943-945

Suki, S. et al. (1995) Leukemia Lymphoma 18(1-2):87-92

Szebeni, J. and Weinstein, J. N. (1991) J. Lab. Clin. Med. 117(6):485-92.

Tannock, I. F. (1996) J. Clin. Oncol. 14(12):3156-3174

Teh, B. T. (1999) Anticancer Res. 19(6A):4715-4728

Tolstikov, V. V. et al. (1997) Nucleosides Nucleotides 16:215-225

Troutner, D. A. (1987) Nucl. Med. Biol. 14:171-176

Tsavaris, N. et al. (1990) J. Chemother. 2(2):123-6

Valette, et al. (1996) J. Med. Chem 39:1981

Voet, et al. (1995) Biochemistry 2^{nd} Ed., John Wiley & Sons, Inc.

Wadler, S. et al. (1987) Cancer Treat Rep. 71(9):821-4.

Wahba, A. J. et al. (1961) J. Biol. Chem. 236(3):C11

Wallis, et al. (1999) Farmaco 54(1-2):83-89

Wataya, Y. (1979) J. Med. Chem. 22:339-340

Wettergren, Y. et al. (1994) Mol. Genet. 20:267-285

Whalen and Boyer (1998) Semin. Liver Dis. 18(4):345-358

Wilson, J. D., et al. (eds.) (1991) “Harrison's Principles of Internal Medicine” 12^{th} ed., McGraw-Hill, Inc., pp. 21-76

Willson, J. K. et al. (1988) Cancer Res. 48(19):5585-90

Wright, A. M. et al. (2000) Leukemia. 14(1):52-60

Yen, Y. et al. (1994) Cancer Res. 54:3686-3691

Zeid, et al. (1999) Nucleosides Nucleotides 18(1):95-111

What is claimed is:

1. A composition comprising a prodrug that is selectively converted to a toxin in the cell by an endogenous, intracellular target enzyme and a nucleoside transport inhibitor.

2. The composition of claim 1, wherein the prodrug is a 1,5-substituted pyrimidine or a substituted furanopyrimidone.

3. The composition of claim 1, wherein the prodrug is a 1,5-substituted pyrimidine.

4. The composition of claim 3, wherein the prodrug is substituted at the 5 position with a group that is extractable from pyrimidine by the endogenous, intracellular enzyme wherein the 5-substituent is selected from the group consisting of alkyl, alkenyl, alkynyl, vinyl, propargyl and substituted derivatives thereof.

5. The composition of claim 3, wherein the 1,5-substituted pyrimidine is substituted at the 1-position with a group selected from substituted sugar, unsubstituted sugar, substituted thio-sugar, unsubstituted thio-sugar, substituted carbocyclic, and unsubstituted carbocyclic.

6. The composition of claim 3, wherein the prodrug is 5-haloalkyl substituted pyrimidine.

7. The composition of claim 3, wherein the prodrug is 5-bromovinyl substituted pyrmidine.

8. The composition of claim 3, wherein the prodrug is a 5′-phosphoryl derivative of pyrimidine.

9. The composition of claim 3, wherein the prodrug is a 5′-phosphoramidate derivative of pyrimidine.

10. The composition of claim 3, wherein the prodrug is (E)-5-(2-bromovinyl)-2′-deoxy-5′-uridyl phenyl L-alaninylphosphoramidate.

11. The composition of claim 1, wherein the nucleoside transport inhibitor is selected from the group consisting of dipyridamole (DP), p-nitrobenzylthioinosine (NBMPR), 6-benzylaminopurine, 2′,3′-dideoxyguanosine, 8-bromoadenine, 9-[(2-hydroxyethoxy)methyl] guanine (Acyclovir), 9-[(1,3-dihydroxy-2-propoxy) methyl] guanine (Ganciclovir), adenine, hypoxanthine, allopurinol, dilazep, cytochalasin B, lidoflaxine, mioflazine, phloretin, phloridzine, and benzylisoquinoline alkaloids.

12. The composition of claim 10, wherein the nucleoside transport inhibitor is selected from the group consisting of dipyridamole (DP), p-nitrobenzylthioinosine (NBMPR), 6-benzylaminopurine, 2′,3′-dideoxyguanosine, 8-bromoadenine, 9-[(2-hydroxyethoxy)methyl] guanine (Acyclovir), 9-[(1,3-dihydroxy-2-propoxy) methyl] guanine (Ganciclovir), adenine, hypoxanthine, allopurinol, dilazep, cytochalasin B, lidoflaxine, mioflazine, phloretin, phloridzine, and benzylisoquinoline alkaloids.

13. The composition of claim 1, wherein the nucleoside transport inhibitor is djipyridamole or p-nitrobenzylthioninosine.

14. The composition of claim 1, wherein the nucleoside transport inhibitor is a benzylisoquinoline alkaloid selected from the group consisting of papaverine, ethaverine, laudanosine, noscarpine, and berberine.

15. A method for inhibiting the growth of a hyperproliferative cells, wherein the cells express an endogenous, overexpressed intracellular target enzyme comprising contacting the cell with an effective amount of the composition of claim 1.

16. The method of claim 15, wherein the contacting is in vitro, ex vivo or in vivo.

17. The method of claim 15, wherein the hyperproliferative cells are resistant to a chemotherapeutic drug.

18. The method of claim 15, wherein the endogenous overexpressed intracellular target enzyme is thymidylate synthase.

19. The method of claim 15, wherein the hyperproliferative cell is a cancer cell.

20. The method of claim 19, wherein the cancer cell is selected from the group consisting of a sarcoma cell, a leukemia cell, a carcinoma cell and an adenocarcinoma cell.

21. The method of claim 19, wherein the cancer cell is selected from the group consisting of a colorectal cancer cell, a head and neck cancer cell, a breast cancer cell, a hepatoma cell, a liver cancer cell, a pancreatic carcinoma cell, an esophageal carcinoma cell, a bladder cancer cell, a gastrointestinal cancer cell, an ovarian cancer cell, a skin cancer cell, a prostate cancer cell, and a gastric cancer cell.

22. A method for treating a subject having a pathology characterized by hyperproliferative cells that are resistant to chemotherapy by the overexpression of an endogenous intracellular enzyme comprising administering to the subject an effective amount of the composition of claim 1.

23. The method of claim 22, further comprising administering to the subject an effective amount of the chemotherapy to which the cells had become resistant.

24. The method of claim 22 or 23, wherein the endogenous intracellular target enzyme is thymidylate synthase.

25. The method of claim 22, wherein the hyperproliferative cell is a cancer cell.

26. The method of claim 25, wherein the cancer cell is selected from the group consisting of a sarcoma cell, a leukemia cell, a carcinoma cell and an adenocarcinoma cell.

27. The method of claim 25, wherein the cancer cell is selected from the group consisting of a colorectal cancer cell, a head and neck cancer cell, a breast cancer cell, a hepatoma cell, a liver cancer cell, a pancreatic carcinoma cell, an esophageal carcinoma cell, a bladder cancer cell, a gastrointestinal cancer cell, an ovarian cancer cell, a skin cancer cell, a prostate cancer cell, and a gastric cancer cell.

28. A method for treating a subject having a pathology characterized by hyperproliferative cells that are resistant to chemotherapy by the overexpression of thymidylate synthase comprising administering to the subject an effective amount of a composition comprising a prodrug that is selectively converted to a toxin in the cell by thymidylate synthase and a nucleoside transport inhibitor.

29. A method for treating a subject having breast cancer characterized by hyperproliferative cells that are resistant to chemotherapy by the overexpression of an endogenous intracellular enzyme comprising administering to the subject an effective amount of the composition of claim 1.

30. A method for treating a subject having colon cancer characterized by hyperproliferative cells that are resistant to chemotherapy by the overexpression of an endogenous intracellular enzyme comprising administering to the subject an effective amount of the composition of claim 1.

31. A method for treating a subject having breast or colon cancer characterized by hyperproliferative cells that are resistant to chemotherapy by the overexpression of thymidylate synthase comprising administering to the subject an effective amount of a composition comprising (E)-5-(2-bromovinyl)-2′-deoxy-5′-uridyl phenyl L-alaninylphosphoramidate and a nucleoside transport inhibitor.

32. A method for treating a subject having breast or colon cancer characterized by hyperproliferative cells that are resistant to chemotherapy by the overexpression of thymidylate synthase comprising administering to the subject an effective amount of a composition comprising (E)-5-(2-bromovinyl)-2′-deoxy-5′-uridyl phenyl L-alaninylphosphoramidate and dipyridamole.

33. A method for treating a subject having breast or colon cancer characterized by hyperproliferative cells that are resistant to chemotherapy by the overexpression of thymidylate synthase comprising administering to the subject an effective amount of a composition comprising (E)-5-(2-bromovinyl)-2′-deoxy-5′-uridyl phenyl L-alaninylphosphoramidate and p-nitrobenzylthioinosine.

34. An assay for selecting agents that enhances the cytotoxicity of a prodrug that is selectively activated by an endogenous intracellular enzyme in hyperproliferative cells comprising contacting a first sample of hyperproliferative cells with an effective amount of the prodrug and the agent to be assayed and contacting a second sample of counterpart normal cells with an effective amount of the agent to be tested and the prodrug and selecting agents that inhibit the proliferation of the first sample of cells but do not inhibit the proliferation of the second sample of cells.

35. The method of claim 34, wherein the hyperproliferative cell is a cancer cell.

36. The method of claim 35, wherein the cancer cell is selected from the group consisting of a sarcoma cell, a leukemia cell, a carcinoma cell and an adenocarcinoma cell.

37. The method of claim 35, wherein the cancer cell is selected from the group consisting of a colorectal cancer cell, a head and neck cancer cell, a breast cancer cell, a hepatoma cell, a liver cancer cell, a pancreatic carcinoma cell, an esophageal carcinoma cell, a bladder cancer cell, a gastrointestinal cancer cell, an ovarian cancer cell, a skin cancer cell, a prostate cancer cell, and a gastric cancer cell.

38. A method to enhance the cytotoxity of an ECTA compound against a cell containing an intracellular target enzyme that is endogenously overexpressed in the cell by contacting the cell with an effective amount of a nucleoside inhibitor compound.

39. The method of claim 38, wherein the nucleoside inhibitor compound is dipyridamole or p-nitrobenzylthioinosine.

40. The method of claim 39, wherein the prodrug is 1,5-substituted pyrimidine.

41. The method of claim 38, wherein the prodrug is substituted at the 5 position with a group that is extractable from pyrimidine by the endogenous, intracellular enzyme wherein the 5-substituent is selected from the group consisting of alkyl, alkenyl, alkynyl, vinyl, propargyl and substituted derivatives thereof.

42. The method of claim 40, wherein the 1,5-substituted pyrimidine is substituted at the 1-position with a group selected from substituted sugar, unsubstituted sugar, substituted thio-sugar, unsubstituted thio-sugar, substituted carbocyclic, and unsubstituted carbocyclic.

43. The method of claim 38, wherein the prodrug is 2-haloalkyl substituted pyrimidine.

44. The method of claim 38, wherein the prodrug is 5-bromovinyl substituted pyrimidine.

45. The method of claim 38, wherein the prodrug is a 5′-phosphoryl derivative of pyrimidine.

46. The composition of claim 38, wherein the prodrug is a 5′-phosphoramidate derivative of pyrimidine.

47. The method of claim 38, wherein the nucleoside transport inhibitor is selected from the group consisting of dipyridamole (DP), p-nitrobenzylthioinosine (NBMPR), 6-benzylaminopurine, 2′,3′-dideoxyguaosine, 8-bromoadenine, 9-[(2-hydroxyethoxy)methyl] guanine (Acyclovir), 9-[(1,3-dihydroxy-2-propoxy) methyl] guanine (Ganciclovir), adenine, hypoxanthine, allopurinol, dilazep, cytochalasin B, lidoflaxine, mioflazine, phloretin, phloridzine, and benzylisoquinoline alkaloids.

48. The method of claim 47, wherein the nucleoside transport inhibitor is a benylisoquinoline alkaloid selected from the group consisting of papaverine, ethaverine, laudanosine, noscarpine, and berberine.

49. A method to enhance the cytotoxity of (E)-5-(2-bromovinyl)-2′-deoxy-5′-uridyl phenyl L-alaninylphosphoramidate against a hyperproliferative cell containing intracellular thymidylate synthase by contacting the cell with an effective amount of dipyridamole.

50. A method to enhance the cytotoxity of (E)-5-(2-bromovinyl)-2′-deoxy-5′-uridyl phenyl L-alaninylphosphoramidate against a cell containing intracellular thymidylate synthase by contacting the cell with an effective amount of p-nitrobenzylthioinosine.

51. A method to enhance the cytotoxity of (E)-5-(2-bromovinyl)-2′-deoxy-5′-uridyl phenyl L-alaninylphosphoramidate against a breast cancer cell containing intracellular thymidylate synthase by contacting the cell with an effective amount of p-nitrobenzylthioinosine.

52. A method to enhance the cytotoxity of (E)-5-(2-bromovinyl)-2′-deoxy-5′-uridyl phenyl L-alaninylphosphoramidate against a colon cancer cell containing intracellular thymidylate synthase by contacting the cell with an effective amount of dipyridamole.
